(19) United States
(12) Patent Application Publication
(10) Pub. No.: US 2010/0284919 A1
(43) Pub. Date: Nov. 11, 2010

(54) Injectable Radio-Opaque Compositions for Tissue Augmentation

(75) Inventors: Enrico Zamparo, Zurich (CH); Laurent Rapillard, Fully (CH); Simona Cerritelli, Zurich (CH); Sauro Bianchi, Zurich (CH)

Correspondence Address: Pabst Patent Group LLP, 1545 PEACHTREE STREET NE, SUITE 320, ATLANTA, GA, 30309, US

(73) Assignee: KUROS BIOSURGERY AG, Zurich (CH)

(21) Appl. No.: 12/682,758

(22) PCT Filed: Oct. 13, 2008
(86) PCT No.: PCT/EP2008/063726

§ 371 Date: Apr. 12, 2010

Related U.S. Application Data
(60) Provisional application No. 60/979,750, filed on Oct. 12, 2007.
(60) Provisional application No. 61/099,083, filed on Sep. 22, 2008.

(51) Int. Cl. A61K 38/48; A61K 49/00; A61P 19/00

(52) U.S. Cl. 424/9.1; 424/94.64

(57) ABSTRACT

Injectable radio-opaque compositions for tissue augmentation and in particular hard tissue augmentation, and kits and methods of using thereof are described herein. The injectable compositions form porous, biologically degradable, fibrin matrices. The compositions are formed from fibrinogen, thrombin or another agent that causes the fibrinogen to crosslink, and strontium salts. Optionally an iodinated contrast agent is further incorporated in the composition. In certain aspects, the compositions have substantially no exothermicity when forming the matrix and the resulting matrices exhibit mechanical properties typically seen in elastomers. Adequate radio-opacity is achieved through the incorporation of strontium salts in combination or not with iodinated contrast agents.

### FIELD OF THE INVENTION

The present invention generally relates to compositions for tissue augmentation, precursor components capable of forming the compositions and to the uses for such compositions. In particular, the present invention relates to the formation of compositions for augmentation of hard tissue.

### BACKGROUND OF THE INVENTION

Percutaneous hard tissue augmentation encompasses medical procedures including vertebroplasty and kyphoplasty. Percutaneous vertebroplasty is a technique in which acrylic cement is injected through a needle into a collapsed or weakened vertebra to stabilize the fracture. This procedure is effective for treating certain types of painful vertebral compression fractures and some painful or unstable benign and malignant vertebral lesions that fail to respond to the traditional conservative therapies (Predley et al. American Family Physician 2002, 66(4): 611-615). Most experts believe that pain relief is achieved through mechanical support and stability provided by cement. The solid mixture of polymethylmethacrylate (PMMA), an acrylic cement used in orthopedic procedures, has been shown to restore strength and stiffness in vertebral bodies in postmortem studies.

The cements used for vertebroplasty have a proven safety record in orthopedics since their first use in the 1960s. Nevertheless, research is still being conducted to develop better injectable hard tissue augmentation materials. Ideally, these substances will strengthen the vertebral body while inducing new hard tissue growth.

Vertebroplasty and kyphoplasty have been used in the following indications: osteoporotic compression fractures, hemangiomas, traumatic compression fractures and vertebral metastasis. The standard material used in vertebroplasty and kyphoplasty is polymethylmethacrylate (PMMA), while other cements (e.g. calcium phosphate cements) are used at minor extent. The procedure involves the percutaneous injection of the cement under fluoroscopic guidance. The cement is injected through a trocar in the area to be treated and cures in situ, thus providing mechanical stability and pain relief. Despite being very efficacious, PMMA has several drawbacks including monomer toxicity, high setting temperature, narrow working time window, higher stiffness than trabecular hard tissue. In particular the latter characteristic is believed to confer too high stiffness to the treated vertebra/ae, which alters the load distribution in the spine and consequently increases the risk of fracture of the adjacent vertebrae (Trout A T, Kallmes D F, Kauffman T J, Journal of Neuroradiology, 2006, 27: 217-223). In order to prevent the adjacent vertebrae from fracturing, PMMA is currently used also as a prophylactic treatment. However, this procedure may only transfer the risk of fracture to other vertebrae.

US 2007/0275028 and US 2007/0276505 disclose biodegradable injectable compositions comprising fibrin for bone augmentation or for use as bone void filler. According to these inventions, the properties (such as the mechanical properties of the material necessary for bone augmentation or for use as a bone void filler) are tuned by adjusting type and content of the particles as well as of the plasticizer contained in the compositions. This means that the plasticizer or the particles present in the compositions are absolutely necessary to achieve the desired effect. The presence of particles or plasticizer in the composition have significant effect on the mechanical characteristics of the fribrin clot formed (C. I. Jones, A. H. Goodall/Thrombosis Research 112 (2003) 65-71, J of Biological Chemistry, vol. 267, No. 34, December 5, pp. 24259-24263, 1992) and the presence of particles could interfere with the analysis of new hard tissue formation in the treated area. Additionally, the presence of particles could represent a safety concern in case of material migration to a site that is not the one indicated for treatment. For example, the presence of particles could be dangerous in case of material leakage and subsequent pulmonary embolism when the material is used for vertebroplasty.

Iodinated contrast agents are known for influencing the clotting process of fibrin. In particular, the presence of iodine-based contrast agents leads to the formation of thin fibers and small pores in the clotted fibrin sealant, which significantly prolong the degradation time of the clot and make the diffusion of added bioactives and the cell infiltration of body's own cells necessary during the process of tissue regeneration more difficult (see Thrombosis Research, 112: 65-71 (2003); J. Biological Chemistry, 267 (34): 24259-24263 (1992)).

A need exists for tissue augmentation compositions which overcome the above-mentioned disadvantages.

Therefore, it is an object of the invention to provide injectable radio-opaque compositions for tissue augmentation wherein the compositions form crosslinked matrices which retain their osteogenic or cell in-growth properties.

It is also an object of the invention to provide improved compositions for hard tissue augmentation wherein the mechanical characteristics suitable for hard tissue augmentation are mainly provided by the mechanical characteristics of the crosslinked matrix per se.

It is an object of the present invention to provide compositions for forming crosslinked matrices wherein the crosslinked matrices as such possess the required mechanical characteristics for use in hard tissue augmentation.

It is a further object of the invention to provide improved kits, methods and uses for tissue augmentation and in particular for hard tissue augmentation.

### SUMMARY OF THE INVENTION

Injectable compositions for tissue augmentation, use of the composition, and kits and methods of using thereof are described herein. The injectable compositions form porous, biologically degradable, fibrin matrices. The injectable compositions are formed from the following precursor components: fibrinogen, thrombin or another agent that causes the fibrinogen to crosslink to form a fibrin clot, and a strontium salt. Optionally, the compositions of the invention further comprise a parathyroid hormone and/or an iodinated contrast agent. In certain aspects, the formation of the matrix from the compositions is substantially not exothermic and the resulting matrices exhibit mechanical properties typically seen in elastomers. Adequate radio-opacity is achieved through the incorporation of strontium salt and/or suitable iodinated contrast agents. Adequate viscosity and clot strength are achieved and controlled by the incorporation of agents in powder form, e.g. strontium salts. Adequate viscosity and clot strength can also be achieved and controlled by the concentration of the fibrinogen component in the final fibrin matrix (also referred to herein as a “fibrin clot”), i.e. by the structure of the fiber mesh that constitutes the fibrin clot. Unlike bone augmentation cements, such as PMMA, the compositions described herein do not have an upper time limit for completion of the technique allowing more time to deliver the material to the site to be treated.

In one embodiment an injectable composition for tissue augmentation and in particular hard tissue augmentation comprises
(a) a first component comprising fibrinogen;
(b) a second component comprising thrombin; and
(c) a third component comprising a strontium salt

Preferably, the strontium salt is strontium carbonate. Preferably, the strontium salt, especially the strontium carbonate is present in the composition from about 0.05 to about 0.60 g strontium salt per ml of volume (a)+(b) (5% to about 60% weight/volume).

In another preferred embodiment, the compositions comprise fibrinogen in an amount from about 25 to about 100 mg fibrinogen/ml of the total volume of the first component and the second component (“volume (a)+(b)”) and wherein the second component comprises from about 1 to about 40 IU thrombin/ml of volume (a)+(b).

In another embodiment, the compositions may further comprise parathyroid hormone or biologically functional fragment thereof. Preferably, the parathyroid hormone is a PTH fusion peptide (TGplPTH₁₋₃₄). More preferably, the compositions comprise from about 0.2 to 5 mg/ml parathyroid hormone of the total volume of the first component and the second component (“volume (a)+(b)”). Optionally, the parathyroid hormone is covalently crosslinked to fibrin produced during clotting of the fibrinogen.

In another embodiment, the compositions of the present invention may further comprise an iodinated contrast agent. Preferably, the iodinated contrast agent is selected from the group consisting of diatrizoate, iodecol, iodixanol, iofratol, iogulamide, iohexyl, iomeprol, iopamidol, iotrol, ioversol, ioxaglate and metrizamide. More preferably, the compositions comprise an iodinated contrast from about 50 and about 500 mg of contrast agent per ml of volume (a)+(b), even more preferably, from about 100 to about 450 mg of contrast agent/ml of volume (a)+(b). Optionally, the iodinated contrast agent is present in the compositions in a dry powdered form.

In another embodiment, a kit comprises:
a) a first container comprising fibrinogen,
b) a second container comprising thrombin, and
c) a third container comprising a strontium salt.
   Preferably, the strontium salt is strontium carbonate, as outlined above.

Optionally, the first container further comprises a parathyroid hormone or biologically functional fragment thereof.

Optionally, the kit further comprises a fourth container comprising a dry, powdered iodinated contrast agent or a iodinated contrast agent in solution.

In other embodiments are provided:

The use of the injectable compositions of the present invention in the manufacture of a medicament for tissue augmentation and in particular hard tissue augmentation.

The compositions of the present invention for use in tissue augmentation and in particular hard tissue augmentation.

The use of the compositions of the present invention in the manufacture of a medicament for preventing or treating a fracture.

The compositions of the present invention for use in the prevention or treatment of a fracture.

### BRIEF DESCRIPTION OF THE DRAWINGS

FIG. 1a shows a line graph of viscosity (Pa s) versus time (seconds) for the following five (5) fibrinogen compositions: (F1) Fibrinogen 57 mg/ml of fibrin, Thrombin 11 IU/ml of fibrin, 400 mg Iopamidol powder per ml of fibrin, 0.1 g 10 micron tricalcium phosphate powder per ml fibrin; (F2) Fibrinogen 57 mg/ml of fibrin, Thrombin 11 IU/ml of fibrin, 400 mg Iopamidol powder per ml of fibrin; (F3) Fibrinogen 40 mg/ml of fibrin, Thrombin 11 IU/ml of fibrin, 400 mg Iopamidol powder per ml of fibrin; (F4) Fibrinogen 57 mg/ml of fibrin, Thrombin 11 IU/ml of fibrin, 400 mg Iohexyl powder per ml of fibrin; (F5) Fibrinogen 40 mg/ml of fibrin, Thrombin 11 IU/ml of fibrin, 400 mg Iopamidol in solution in the thrombin syringe.

FIG. 1b is a graph of Viscosity (PaS) versus time (seconds) for two compositions, Composition F1 (no PEG) (Solid line) and Composition F2 (with PEG) (dashed line).

FIGS. 2a and 2b show diagrams of instrumentation used to determine extrusion parameters of various fibrinogen compositions.

FIG. 3 is a line graph showing bioactive release over time for the following seven (7) fibrinogen compositions: (FA) fibrinogen 45 mg per ml of fibrin, thrombin 2 IU per ml of fibrin; (FB) fibrinogen 54 mg per ml of fibrin, thrombin 4 IU per ml of fibrin, 300 mg Iodixanol per ml fibrin (Iodixanol in solution stored separately from fibrinogen and thrombin and added to fibrinogen/thrombin mixture once the two have been mixed together); (FC) fibrinogen 45 mg per ml of fibrin, thrombin 37.5 IU per ml of fibrin, 300 mg Iodixanol per ml fibrin (Iodixanol in solution together with thrombin component); (FD) fibrinogen 45 mg per ml of fibrin, thrombin 37.5 IU per ml of fibrin, 400 mg Iodixanol per ml fibrin (Iodixanol in solution together with thrombin component); (FE) fibrinogen 44 mg per ml of fibrin, thrombin 11 IU per ml of fibrin, 303 mg Iodixanol per ml fibrin (Iodixanol in solution stored separately from fibrinogen and thrombin and added to fibrinogen/thrombin mixture once the two have been mixed together); (FF) fibrinogen 48 mg per ml of fibrin, thrombin 4 IU per ml of fibrin, 300 mg Iohexyl per ml fibrin (Iohexyl in solution stored separately from fibrinogen and thrombin and added to fibrinogen/thrombin mixture once the two have been mixed together); (FG) fibrinogen 53 mg per ml of fibrin, thrombin 10 IU per ml of fibrin, 400 mg Iopamidol in powder per ml fibrin.

FIG. 4a and FIG. 4b show the compressive elastic modulus for the following formulations: 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 60 mg/ml of fibrin, Thrombin 7 IU/ml of fibrin, Iodixanol 450 mg/ml of fibrin in powder form; 5) Fibrinogen 56 mg/ml of fibrin, Thrombin 2 IU/ml of fibrin, strontium carbonate 40% w/v powder (added to the mixture after the fibrinogen and thrombin components have been mixed); 6) Fibrinogen 42 mg/ml, thrombin 41 U/ml, iodixanol 150 mg/ml pre-dissolved in the thrombin solution, strontium carbonate 25% w/v in powder; 7) Fibrinogen 42 mg/ml, thrombin 21 U/ml, iodixanol 400 mg/ml pre-dissolved in the thrombin solution, tri-calcium phosphate 25% w/v in powder.

FIG. 5a and FIG. 5b show the deformation energy at 50% strain following uniaxial compression testing for the following formulations: 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 60 mg/ml of fibrin, Thrombin 7 IU/ml of fibrin, Iodixanol 450 mg/ml of fibrin in powder form; 5) Fibrinogen 56 mg/ml of fibrin, Thrombin 2 IU/ml of fibrin, strontium carbonate 40% w/v powder (added to the mixture after the fibrinogen and thrombin components have been mixed); 6) Fibrinogen 42 mg/ml, thrombin 41 U/ml, iodixanol 150 mg/ml pre-dissolved in the thrombin solution, strontium carbonate 25% w/v in powder; 7) Fibrinogen 42 mg/ml, thrombin 21 U/ml, iodixanol 400 mg/ml pre-dissolved in the thrombin solution, tri-calcium phosphate 25% w/v in powder.

FIG. 6a shows a line graph of the amount of buffer separated after extrusion through a 10 G 100 mm vertebroplasty needle; FIG. 6b shows a line graph of the amount of volatile components lost after exposure of the injected material for 5 h at 37° C.; FIG. 6c shows a line graph of the percent weight loss after exposure of the injected material for 5 h at 37° C. for the following formulations: 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 84 mg/ml of fibrin, Thrombin 2 IU/ml of fibrin; 5) Fibrinogen 60 mg/ml of fibrin, Thrombin 7 IU/ml of fibrin, Iodixanol 450 mg/ml of fibrin in powder form; 6) Fibrinogen 56 mg/ml of fibrin, Thrombin 2 IU/ml of fibrin, strontium carbonate 40% w/v powder (added to the mixture after the fibrinogen and thrombin components have been mixed).

FIGS. 7a and 7b show buffer release following uniaxial compression testing for the following formulations: 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 84 mg/ml of fibrin, Thrombin 2 IU/ml of fibrin; 5) Fibrinogen 60 mg/ml of fibrin, Thrombin 7 IU/ml of fibrin, Iodixanol 450 mg/ml of fibrin in powder form; 6) Fibrinogen 56 mg/ml of fibrin, Thrombin 2 IU/ml of fibrin, strontium carbonate 40% w/v powder (added to the mixture after the fibrinogen and thrombin components have been mixed); 7) Fibrinogen 42 mg/ml, thrombin 41 U/ml, iodixanol 150 mg/ml pre-dissolved in the thrombin solution, strontium carbonate 25% w/v in powder; 8) Fibrinogen 42 mg/ml, thrombin 21 U/ml, iodixanol 400 mg/ml pre-dissolved in the thrombin solution, tri-calcium phosphate 25% w/v in powder.

FIG. 8a is a graph of % release of the bioactive agent from each of four compositions over time (hours). Composition Fa (♦), Composition Fb (▪), Composition Fc (▴), and Composition Fd (x).

FIG. 8b is a graph of % release of the bioactive agent from each of three compositions over time (hours). Composition Fa (♦), Composition Fb (▴), and Composition Fc (▪).

FIG. 9 shows a bar graph of the % of bone volume over total volume in sheep vertebrae after treatment with the following formulations: A) Fibrinogen 60 mg/ml, thrombin 8 IU/ml, Iopamidol (powder) 425 mg/ml, TGplPTH1-34 0.4 mg/ml. Injected into the third lumbar vertebra (L3); B) Fibrinogen 60 mg/ml, thrombin 8 IU/ml, Iodixanol (powder) 425 mg/ml, TGplPTH1-34 0.4 mg/ml. Injected into L2; C) Fibrinogen 60 mg/ml, thrombin 8 IU/ml, Iodixanol (powder) 425 mg/ml, TGplPTH1-34 0.4 mg/ml. Injected into L5; D) Fibrinogen 42 mg/ml, thrombin 8 IU/ml, Iodixanol (powder) 425 mg/ml, 10 micron tricalcium phosphate 15% w/v, TGplPTH1-34 0.4 mg/ml. Injected into L4.

FIG. 10 shows the custom aiming device manufactured for surgery in the femur condyle/proximal tibia augmentation model in the sheep.

FIG. 11a and 11b show the positioning of the aiming device under fluoroscopy and the position of the injected material within the trabecular spacing of existing intact bone.

FIG. 12 shows μCT slices covering a total thickness of about 8.1 mm showing dense bone formation around the tip of the needle growing towards the center of the cloud. Residual strontium carbonate is still present (Formulation 3, right tibia).

FIG. 13 shows the calculation of bone volume/total volume (BV/TV) in spherical volumes of interest centered on the original position of the injection needle tip indicating a positive effect of all the Vertebral Augmentation Material formulations with respect to bone formation.

### DETAILED DESCRIPTION OF THE INVENTION

### I. Definitions

The term “active agent” or “drug” or “bioactive agent” as generally used herein refers to a compound which affects or modifies a biological process. Active agents are used for the treatment, prevention, or diagnosis (e.g., therapeutic, diagnostic, and prophylactic agents) of a disease or disorder in an animal, such as a human.

The term “clotted” as generally used herein means, for example, a gel comprising fibrin and includes any kind of coagulation state known in the art.

The term “fibrinogen” includes not only fibrinogen per se, but also clot-forming derivatives of fibrinogen.

As generally used herein, the term “thrombin” includes thrombin per se, as well as any gelation-inducing or clot-inducing agent for fibrinogen or fibrin.

As generally used herein, the term “gelled” means any state of elevated viscosity when compared to the initial state. This can be observed for example in the formation of fibrin from fibrinogen or in a finely dispersed system of at least one solid phase and at least one liquid phase, such as a colloid. Further, the term “gelled” includes all states of gelation known in the art.

The term “hard tissue” as generally used herein refers to bone, disc, tendon, ligament and cartilage.

The term “matrix” as generally used herein refers to a material intended to interface with biological systems to treat, augment, or replace any tissue or function of the tissue depending on the material either permanently or temporarily. The matrix can serve as a delivery device for active agents incorporated therein. In one embodiment, the matrices described herein are formed from liquid precursor components which are able to form a gel in the body at the site of need. The terms “matrix”, “gel”, “sealant” and “three-dimensional network” are used synonymously herein. The terms “matrix”, “gel” and “sealant” refer to the composition formed after the precursor components are mixed together and the crosslinking reaction has begun. Thus the terms “matrix”, “gel” and “sealant” encompass partially or fully crosslinked polymeric networks. They may be in the form of a semi-solid, such as a paste, or a solid. Depending on the type of precursor materials, the matrix may be swollen with water but not dissolved in water, i.e. form a hydrogel, which stays in the body for a certain period of time.

The term “fibrin matrix”, as generally used herein, refers to a three-dimensional matrix formed from precursor components containing fibrinogen and thrombin, which crosslink in the presence of a calcium source, Factor XIIIa and optionally, one or more excipients present in the precursor components.

The terms “bone fracture” as used herein refers to a discontinuity or break across the entire bone structure creating two or more distinct bone segments.

### II. Injectable Compositions for Tissue Augmentation

One embodiment provides an injectable tissue augmentation composition and in particular an injectable hard tissue augmentation composition including at least a first component, a second component and a third component. The first component, referred to herein as “component (a)”, includes fibrinogen or fibrin. The second component, referred to herein as “component (b)”, includes thrombin or another agent that causes the first component to crosslink, or form a clot or matrix. The third component, sometimes referred to herein as “component (c)”, includes a strontium salt. Preferably, the strontium salt is strontium carbonate (SrCO₃). This composition may be used in the cases where there is a need to visualize the material with x-rays during and after the procedure.

The composition optionally includes a parathyroid hormone or biologically functional fragment thereof. Preferably the parythyroid hormone is a PTH fusion protein (TGplPTH₁₋₃₄).

The composition optionally further includes an iodinated contrast agent.

In some embodiments, the hard tissue augmentation compositions contain particulate fillers, such as calcium salt particles.

The resulting tissue augmentation compositions are fully resorbable by the body and are biodegradable.

Generally, fibrinogen and thrombin are present in solution prior to mixing. The strontium slat is provided in solid powder spatially separated from the other components, prior to mixing.

The injectable tissue augmentation compositions advantageously allow injection into a non-mineralized or hollow portion of a tissue while the procedure can be contemporaneously monitored by a variety of imaging methods such as fluoroscopy. Moreover, water soluble contrast agents can diffuse out of the composition almost completely after one week. Other beneficial characteristics of the compositions include the highly advantageous biodegradability of the final clotted composition, the ease of handling and long term storage stability and high availability, the radio-opacity and viscosity.

In one embodiment, the additional contrast agent of the multi-component system includes at least one iodine containing organic compound.

The injectable tissue augmentation compositions can be in a gelled or partially crosslinked state and have a viscosity suitable for injecting into a non-mineralized or hollow portion of a tissue, for example a trabecular bone. The compositions may be applied in a pre-clotted liquid, gelled or clotted state.

The viscosity or flow properties of the injectable compositions can be varied depending on the application of the compositions. Typically, the flow properties are adjusted by changing the ratio between components (a), (b) and (c), and in particular by varying the concentration of fibrinogen. As generally used herein “Volume (a)+(b)” refers to the total volume when component (a) comprising fibrinogen is mixed with component (b) comprising thrombin. The higher the concentration of fibrinogen, the more viscous the composition and the higher the strength of the final fibrin clot. For example, higher concentrations of fibrinogen can be suitable for mechanically challenging indications like disc, tendon and ligament repair. Moreover, the higher the concentration of fibrinogen, the more resistant the clot is. Higher concentrations of fibrinogen also allow for less buffer separation during injection (as described in Example 3). The viscosity can also be adjusted by the presence strontium salts in powder form that at the same time contribute to the radio-opacity of the material.

A. Fibrinogen

In one embodiment, the amount of fibrinogen in the final fibrin clot ranges for example from about 25 to about 100 mg/ml, typically from about 30 to about 80, and even more typically from about 45 to about 70 mg/ml, more preferably from about 50 to about 66 mg/ml, even more preferably from about 53 to about 60 mg/ml, even more preferably from about 54 to about 57 mg/ml and most preferably from about 55 mg/ml. This can be obtained, for example by using component (a) at a concentration of 100 mg/ml fibrinogen and mix it with component (b), for example in volume ratios volume (a):volume (b)=2 (final concentration of fibrinogen in fibrin=66.7 mg/ml) to volume ratios volume (a)/volume (b)=4 (final concentration of fibrinogen in fibrin=80 mg/ml). Alternatively, the same result can be obtained by keeping volume ratios volume (a):volume (b)=1 and using concentrations of fibrinogen in component (a) for example ranging from about 100 to 200 mg/ml, such as from about 114 mg/ml to 160 mg/ml.

The amount of fibrinogen in the Volume (a)+(b) can be from about 25 to 100 mg/ml, and the amount of thrombin in Volume (a)+(b) can be from 1 to about 40 IU/ml. The amount of strontium carbonate can be from 0.05 to 0.60 g per ml of Volume (a)+(b) (hereinafter indicated as 0.5% to 60% weight/volume, where 1% w/v represent 0.01 g contrast agent per ml of Volume (a)+(b)).

In a specific embodiment, the amount of fibrinogen in the Volume (a)+(b) can be from about 45 to about 70 mg/ml, preferably from about 50 to about 66 mg/ml, more preferably from about 53 to about 60 mg/ml, even more preferably from about 54 to about 57 mg/ml and most preferably from about 55 mg/ml and the amount of thrombin in the Volume (a)+(b) can be from 2 IU/ml to about 15 IU/ml. The higher the concentration of fibrinogen, the stronger the fibrin clot and the less buffer separation during injection under pressure.

B. Thrombin

Thrombin (activated Factor II, also referred to as “Factor IIa”) is a coagulation protein that has many effects in the coagulation cascade. It is a serine protease that converts soluble fibrinogen into insoluble strands of fibrin, as well as catalyzing many other coagulation-related reactions. Instead of or in addition to thrombin, Component (b) may contain other fibrinogen-crosslinking compounds known in the art.

Thrombin or component (b) of the multi-component system may further comprise additional compounds known in the art. The concentration of thrombin in component (b) can vary such that in the total volume (a)+volume (b) (“Volume (a)+(b)”) is between 1 and about 40 IU/ml, typically is between 2 and 20 IU/ml and more preferably between 2 and 15 IU/ml.

C. Strontium Salts

Strontium salts, on their own, possess contrast properties. This may allow one to use no iodinated contrast agent or a reduced amount of iodinated contrast agent to obtain optimal contrast for an image guided application. It has surprisingly be found that incorporation of strontium salts into fibrin matrices do not alter the fibrin clot structure and that the fibrin matrices while being radio-opaque retain their osteogenic and cell in-growth properties. The partial or total elimination of the iodine based contrast agent avoids the alteration of the fibrin clot structure. In addition, inorganic salts as strontium carbonate may have a positive effect on bone formation.

Strontium salts include but are not limited to inorganic complexes for example strontium carbonate (SrCO₃), strontium bromide, strontium phosphate (Sr₃(PO₄)₂), strontium hydrophosphate (SrHPO₄), strontium chloride (SrCl₂), strontium acetate (Sr(CH₃COO)₂.1/2H₂O), strontium oxalate SrC₂O₄ and organometallic complexes for example strontium ranelate and strontium citrate. Strontium salts which are known in the art to have a positive effect on bone formation (i.e. osteogenic, osteoconductive or osteoinductive strontium salts) or to have a biological effect on osteoblasts or osteoclasts can also be used in the compositions of the present invention.

In one embodiment, a component (c) contains strontium salt in powder form in an amount ranging from about 0.05 to about 0.60 g strontium salt per ml of volume (a)+(b) (5% to about 60% weight/volume) and more preferably from 10% to 45% weight/volume of the mixture of Volume (a)+(b). Preferably the strontium salt is strontium carbonate.

Further, the strontium salt may be used to improve the flow characteristics of the injectable composition prior to clotting, as well as the strength and the contrast of the final clot. For example, including strontium carbonate in the composition in an amount of 0.1 g/ml of the total volume (a)+(b) including any compounds dissolved therein, such as PEG, but excluding any different powdered components if powdered components are added separately, confers contrast similar to a composition containing 100 mg/ml Iodixanol.

In another embodiment, the preferred composition of the present invention comprises component (a) comprising an amount of fibrinogen in the Volume (a)+(b) from about 45 to about 70 mg/ml, preferably from about 50 to about 66 mg/ml, more preferably from about 53 to about 60 mg/ml, even more preferably from about 54 to about 57 mg/ml and most preferably from about 55 mg/ml; component (b) comprising an amount of thrombin in the Volume (a)+(b) from 2 IU/ml to about 15 IU/ml and an inorganic salt is provided as component (c), in an amount ranging from about 0.05 to about 0.60 g inorganic salt per ml of volume (a)+(b) (5% to about 60% weight/volume) and more preferably from 10% to 45% weight/volume of the mixture of Volume (a)+(b). Preferably the inorganic salt of component (c) is a strontium salt and more preferably the strontium salt is strontium carbonate.

Optionally an inorganic filler may be included in the injectable composition. Suitable fillers include, but are not limited to calcium and strontium salts.

In one embodiment, the composition contains filler in an amount ranging from about 0.01 g/ml to about 0.5 g/ml of the combined volume of fibrinogen and thrombin solutions including any compounds dissolved therein, such as PEG, but excluding any different powdered components if powdered components are added separately. Preferably the composition contains filler in an amount ranging from about 0.05 g/ml and 0.3 g/ml of the combine solutions.

D. Iodinated Contrast Agent

Optionally, the injectable compositions of the present invention further contain an organic contrast agent, preferably an iodinated contrast agent. Suitable organic contrast agents include any iodinated contrast agent usable in imaging applications known in the art such as fluoroscopy. The iodinated contrast agent is useful to distinguish surrounding tissue by detecting and or applying radiation such as X-ray radiation, radioactive radiation, infrared radiation, ultra-violet radiation, electron or neutron radiation, or a magnetic field, ultrasound, or a combination thereof. In one embodiment, the iodinated contrast agent has a low osmolality and allows fibrin assembly to occur to an appropriate extent. Appropriate contrast can be obtained by the use of any combinations of the iodine based contrast agents.

Exemplary iodine based contrast agents include but are not limited to diatrizoate, iodecol, iodixanol, iofratol, iogulamide, iohexyl, iomeprol, iopamidol, iotrol, ioversol, ioxaglate and metrizamide.

The effect of iodine based contrast agents on the properties of fibrin is described in the literature. In particular the presence of iodine based contrast agents, when used in a liquid form, leads to the formation of thin fibers and small pores, which significantly prolong the degradation time of the clot and make the diffusion of macromolecules more difficult (C. I. Jones, A. H. Goodall/Thrombosis Research 112 (2003) 65-71, J of Biological Chemistry, vol. 267, No. 34, December 5, pp. 24259-24263, 1992). The fibrin clot with characteristics altered by the presence of the iodine may be less suitable for use in tissue augmentation and repair.

In a preferred embodiment, the iodine based contrast agent is used in powder form, and is stored separately from fibrinogen and thrombin. The dry contrast agent is typically added to the composition after fibrinogen or fibrin and thrombin have been homogenized. The addition of iodine in powder form to the composition minimizes its effect on the clotting process (as shown in the Examples provided herein). Once mixed with the other components, it distributes homogenously in the composition, thus providing suitable radio-opacity. The mixing of fibrinogen with thrombin prior to adding the powdered contrast agent, allows the clotting of fibrin to start before the iodine is added to the composition.

In one embodiment, the composition contains iodixanol, iohexyl, or iopamidol, in powder form, in an amount ranging from about 50 to about 500 mg per ml of the mixture of Volume (a)+(b), preferably from about 100 to about 450 mg/ml of the mixture of Volume (a)+(b).

In another embodiment, the appropriate contrast is achieved by combinations of iodine and strontium salts. For example, 10% w/v strontium carbonate provides radio-opacity similar to 100 mg/ml Iodixanol.

E. Polyoxyalkylene molecules

The presence of polyoxyalkylenes in the composition was shown (as described in the Examples) to prevent the iodinated contrast agent from having a deleterious effect on the biochemistry of the fibrin clot process and the final structure of the fibrin matrix. The combination of polyoxyalkylenes plus iodinated contrast agent confers to the injectable composition optimal contrast characteristics for image guided application, as well as an open pore and fibrous structure suitable for diffusion of bioactives from the clot and cell infiltration of body's own cells during the process of tissue regeneration.

Generally, the fibrinogen and thrombin components are present in solution prior to mixing. The iodinated contrast agent can be provided in solution with the thrombin component and/or polyoxyalkylene or can be provided in the form of a solid powder that is spatially separated from the other components prior to mixing. The polyoxyalkylene can be provided in solution with thrombin component and/or contrast agent. The filler, if included in the injectable composition, can be provided in the form of a solid powder form that is spatially separated from the other components, prior to mixing or in the form of a solid powder that is premixed with one or more of the components.

Exemplary polyoxyalkylene molecules include but are not limited to non-functionalized, low to high molecular weight, linear or branched polymers. Preferably the polyoxyalkylene is a polyethyleneglycol (PEG).

In one embodiment, the polyoxyalkylene is a linear hydroxy terminated PEG with molecular weights ranging from 1,000 Da to 20,000 Da.

In another embodiment, the polyoxyalkylene is a branched hydroxy terminated PEG with molecular weights ranging from 1,000 Da to 20,000 Da. The hydroxy groups terminate at least two of PEG molecules' arms, preferably all of its arms. The most preferred molecule is a four-arm PEG, with a molecular weight of about 10,000 Da in which all of its arms are hydroxyterminated.

The optimal amount of PEG in the injectable composition varies with the concentration of contrast agent. The amount of PEG in the injectable composition typically ranges from about 5 mg/ml to about 40 mg/ml of the combined volume of fibrinogen and thrombin solutions including any compounds dissolved therein, such as PEG, but excluding any powdered components if powdered components are added separately. The amount of PEG in the injectable composition is preferably from about 10 mg/ml to about 20 mg/ml of the combined volume of fibrinogen and thrombin solutions.

F. Additional Agents

The injectable compositions and precursor components used to form the compositions can optionally include any other component suitable for augmenting, strengthening, supporting, repairing, rebuilding, healing or filling a tissue. Exemplary additional agents that may be used include but are not limited to, calcium salts, such as tricalcium phosphate (TCP) or hydroxyapatite (HA) or mixtures thereof, osteoinductive agents, growth factors such as but not limited to Bone morphogenic protein (BMP-2, BMP-7 or OP-1), transforming growth factor alpha and beta (TGF-α and TGF-β), platelet derived growth factor (PDGF), chemotherapeutic or pharmacological agents, biologically active agents, hardening and/or adhesive compounds and mineral additives.

PTH

The term “PTH” as used herein includes the human sequence of PTH 1-84 and all truncated, modified and allelic versions of PTH which exhibit bone formation properties when covalently bound to biodegradable natural or synthetic matrices. Preferred truncated versions of PTH are PTH 1-38, PTH 1-34, PTH 1-31 or PTH 1-25. Most preferred is PTH 1-34. Preferably, the PTH is human PTH, although PTH from other sources, such as bovine PTH, may be suitable.

“PTH fusion peptide” as generally used herein refers to a peptide which contains at least a first and a second domain. One domain contains a PTH (native or truncated forms, in particular PTH 1-34) and the other domain contains a substrate domain for being crosslinked to a matrix. An enzymatic or hydrolytic degradation site can also be present between the first and the second domain.

The PTH fusion peptide can be crosslinked and covalently bound to matrices through the crosslinkable substrate domain of the PTH fusion peptide. The kind of substrate domain is dependent on the nature of the matrix. For the incorporation into fibrin matrices transglutaminase substrate domains are particularly preferred. The transglutaminase substrate domain may be a Factor XIIIa substrate domain. This Factor XIIIa substrate domain may be or include GAKDV (SEQ ID NO: 1), KKKK (SEQ ID NO: 2), or NQEQVSPL (SEQ ID NO: 3). The coupling between the PTH and the transglutaminase substrate domain can be performed by chemical synthesis.

The transglutaminase substrate domain can be a substrate for a transglutaminase other than Factor XIIIa. The most preferred Factor XIIIa substrate domain has an amino acid sequence of NQEQVSPL (SEQ ID NO: 3) (herein referred to as “TG”). Other proteins that transglutaminase recognizes, such as fibronectin, could be coupled to the transglutaminase substrate peptide.

TABLE 1
Transglutaminase substrate domains

| | |
|---|---|
| SEQ ID NO: 1 | GAKDV |
| | A peptide that mimics the lysine |
| | coupling site in the chain of |
| | fibrinogen |
| SEQ ID NO: 2 | KKKK |
| | A peptide with a polylysine at a |
| | random coupling site |
| SEQ ID NO: 3 | NQEQVSPL |
| | A peptide that mimics the |
| | crosslinking site in α2-plasmin |
| | inhibitor (abbreviated TG) |
| SEQ ID NO: 4 | YRGDTIGEGQQHHLGG |
| | A peptide with glutamine at the |
| | transglutaminase coupling site in the |
| | chain of fibrinogen |

Thus the PTH fusion peptides, may be further modified to contain a degradable site between the attachment site, i.e. the second domain (i.e. factor XIIIa substrate domain or the cysteine) and the PTH, i.e. the first domain. These sites may be degradable either by non-specific hydrolysis (i.e. an ester bond) or they may be substrates for specific enzymatic (either proteolytic or polysaccharide degrading) degradation.

Enzymes that could be used for proteolytic degradation are numerous. Proteolytically degradable sites could include substrates for collagenase, plasmin, elastase, stromelysin, or plasminogen activators. Exemplary substrates are listed below. P1-P5 denote amino acids 1-5 positions toward the amino terminus of the protein from the site were proteolysis occurs. P1′-P4′ denote amino acids 1-4 positions toward the carboxy terminus of the protein from the site where proteolysis occurs.

TABLE 2
Sample substrate sequences for protease.

| | | | | | | | | | | |
|---|---|---|---|---|---|---|---|---|---|---|
| Protease | P5 | P4 | P3 | P2 | P1 | P1′ | P2′ | P3′ | P4′ | Reference |

| | | | | | | | | | | |
|---|---|---|---|---|---|---|---|---|---|---|
| Plasmin | | | L | I | K | M | K | P | | Takagi and Doolittle, (1975) Biochem. |
| | | | | | | | | | | 14: 5149-5156 |
| Plasmin | | | N | F | K | S | Q | L | | Takagi and Doolittle, 1975 |
| Stromelysin | Ac | G | P | L | A | L | T | A | L | Smith et al., (1995). J. Biol. Chem. 270: 6440-6449 |
| Stromelysin | | Ac | P | F | E | L | R | A | NH₂ | Smith et al., 1995 |
| Elastase | | | Z- | A | A | F | A | NH₂ | | Besson et al., (1996) Analytical Biochemistry |
| | | | | | | | | | | 237: 216-223. |
| Collagenase | | G | P | L | G | I | A | G | P | Netzel-Arnett et al., (1991) J. Biol. Chem.. |
| | | | | | | | | | | 266: 6747-6755 |
| t-PA | P | H | Y | G | R | S | G | G | | Coombs et al., 1998. J. Biol. Chem. |
| | | | | | | | | | | 273: 4323-4328 |
| u-PA | P | G | S | G | R | S | A | S | G | Coombs et al., 1998 |

For incorporation of a PTH within the matrix, the matrix includes fibrin which is formed from fibrinogen, a calcium source and thrombin and the PTH fusion peptide will be incorporated within fibrin during coagulation. PTH fusion peptide is designed as fusion peptide which includes two domains, a first and a second one, one domain, the second one, is a substrate for a crosslinking enzyme such as Factor XIIIa. Factor XIIIa is a transglutaminase that is active during coagulation. This enzyme, formed naturally from factor XIII by cleavage by thrombin, functions to attach fibrin chains to each other via amide linkages, formed between glutamine side chains and lysine side chains. The enzyme also functions to attach other peptides to fibrin during coagulation, e.g. the cell attachment sites provided they include a factor XIIIa, too. Specifically the sequence NQEQVSPL (SEQ ID NO: 3), has been demonstrated to function as an effective substrate for factor XIIIa. As described herein before it is either directly linked to the PTH or it can include a degradation site between the PTH (first domain) and the NQEQVSPL (SEQ ID NO: 3) sequence (second domain). As such, the PTH fusion peptide may be incorporated within fibrin during coagulation via a factor XIIIa substrate.

The PTH fusion peptide which includes a first domain including the PTH, a second domain including a substrate domain for a crosslinking enzyme and optionally a degradation site between the first and the second domain can be incorporated into the fibrin gels using several different schemes. Preferably the second domain includes a transglutaminase substrate domain and even more preferably it includes a Factor XIIIa substrate domain. Most preferably the Factor XIIIa substrate domain includes NQEQVSPL (SEQ ID NO: 3). When this PTH fusion peptide is present during the polymerization of the fibrinogen, i.e. during formation of the fibrin matrix, it is directly incorporated into the matrix.

The degradation site between the first and the second domain of the PTH fusion peptide can be an enzymatic degradation site as described previously. Preferably the degradation site is cleavable by an enzyme selected from the group consisting of plasmin and matrix metalloproteinase. By careful selection of Kₘ and k_{cat} of this enzymatic degradation site, degradation could be controlled to occur either before or after the protein matrix and/or by utilizing similar or dissimilar enzymes to degrade the matrix, with the placement of the degradation site being tailored for each type of protein and application. This PTH fusion peptide could be directly cross-linked into the fibrin matrix as described above. However, incorporating an enzymatic degradation site alters the release of the PTH during proteolysis. When the cell-derived proteases reach the sequestered fusion peptide, they can cleave the engineered protein at the newly formed degradation site. The resulting degradation products would include the liberated PTH, which would now be nearly free of any engineered fusion sequences, as well as any degraded fibrin.

In a preferred embodiment, a parathyroid hormone (PTH) or biologically functional fragment thereof, preferably PTH 1-34, is included in the composition. More preferably a PTH fusion peptide which includes a first domain including the PTH, a second domain including a substrate domain for a crosslinking enzyme and optionally a degradation site between the first and the second domain is included in the compositions. Preferably the PTH fusion peptide comprises a first domain including PTH1-34 a second domain which is a transglutaminase substrate domain of SEQ ID No 3 and a plasmin degradation site (TGplPTH₁₋₃₄), as disclosed in U.S. Pat. No. 7,247,609, PCT application WO 03/052091 and U.S. Pat. No. 2,007,0010440, the content of which are incorporated by reference.

In a preferred embodiment, the sequence YKNR (SEQ. NO: 5) is located between the first domain and the second domain and makes the linkage plasmin degradable.

A particular preferred PTH fusion peptide is TGplPTH:

(SEQ ID NO: 6)

NQEQVS-PLYKNRSVSEIQLMHNLGKHLNSMERVEWLRKKLQDVHNF

Preferred fusion proteins include:

TG-PTH 1-34: This is a modified form of PTH comprising the amino acids 1-34 of the native PTH as well as a TG (transglutaminase) substrate domain:

(SEQ ID NO: 7)

NQEQVSPLSVSEIQLMHNLGKHLNSMERVEWLRKKLQDVHNF

Even more preferably, the PTH fusion peptide is covalently crosslinked to fibrin produced during clotting of the fibrinogen. In a preferred embodiment, the compositions comprise PTH or biologically active functional fragment in an amount ranging from about 0.2 to 5 mg/ml parathyroid hormone of the total volume of the first component and the second component (“volume (a)+(b)”).

These compounds may be contained in any of the components (a) to (c) or may be comprised as extra components.

In another embodiment, the fibrinogen component (component (a)), as defined above, may further comprise one or more extracellular matrix proteins, for example fibronectin, cellular associated proteins, other plasma derived proteins, for example blood clotting factor XIII (FXIII) and proteases, and proteases inhibitors, and mixtures thereof. The fibrinogen solution according to the present invention may also include any additive which is comprised in the state of the art for scientific and/or commercially available fibrinogen compositions, for example commercially available fibrinogen solutions.

G. Fibrin Matrices Formed by Injectable Compositions

The injectable compositions for tissue augmentation form fibrin matrices by crosslinking the precursor molecules to form a polymeric network having sufficient inter-polymer spacing to allow for in-growth or migration into the matrix of cells. It has surprisingly been found that the incorporation of strontium salts into the fibrin matrices does not alter the structure of the clot. The resulting compositions have good elasticity and resistance to tension forces.

Fibrin Matrices

Fibrin is a natural material which has been reported for several biomedical applications. Fibrin has been described as material for cell in-growth matrices in U.S. Pat. No. 6,331,422 to Hubbell et al. Fibrin gels have been used as sealants because of its ability to bind to many tissues and its natural role in wound healing. Additionally, these materials have been used as drug delivery devices, and for neuronal regeneration. Fibrin provides a solid support for tissue regeneration and cell in-growth.

The process by which fibrinogen is polymerized into fibrin is well-characterized. Initially, a protease cleaves the dimeric fibrinogen molecule at the two symmetric sites. There are several possible proteases than can cleave fibrinogen, including thrombin, peptidase, and protease III, and each one severs the protein at a different site. Once the fibrinogen is cleaved, a self-polymerization step occurs in which the fibrinogen monomers come together and form a non-covalently crosslinked polymer gel. This self-assembly happens because binding sites become exposed after protease cleavage occurs. Once they are exposed, these binding sites in the centre of the molecule can bind to other sites on the fibrinogen chains, which are present at the ends of the peptide chains. In this manner, a polymer network is formed. Factor XIIIa, a transglutaminase activated from Factor XIII by thrombin proteolysis, may then covalently crosslink the polymer network. Other transglutaminases exist and may also be involved in covalent crosslinking and grafting to the fibrin network.

Once a crosslinked fibrin matrix is formed, the subsequent degradation is tightly controlled. One of the key molecules in controlling the degradation of fibrin is α2-plasmin inhibitor. This molecule acts by crosslinking to the α chain of fibrin through the action of Factor XIIIa. By attaching itself to the gel, a high concentration of inhibitor can be localized to the gel. The inhibitor then acts by preventing the binding of plasminogen to fibrin and inactivating plasmin. The α2-plasmin inhibitor contains a glutamine substrate. The exact sequence has been identified as NQEQVSPL (SEQ ID NO: 3), with the first glutamine being the active amino acid for crosslinking.

Preferably the fibrin matrix is substantially free of a calcium ion source. In one embodiment, the fibrin matrix contains a strontium salt. Preferably, the strontium slat is strontium carbonate. Optionally, the fibrin matrix contains an active agent, such as PTH and/or an iodinated contrast agent.

### III. Kits

In one embodiment, a kit comprises:
a) a first container comprising fibrinogen;
b) a second container comprising thrombin; and
c) a strontium salt, in particular being comprised in a third container.

Preferably, the first container comprises from about 25 to about 100 mg fibrinogen/ml of the total volume of the first container and the second container (volume (a)+(b)) and the second container comprises from about 1 to about 40 IU thrombin/ml of volume (a)+(b). Preferably, a third container (c) comprises from about 0.05 to 0.6 g strontium carbonate per ml or the total volume of components (a)+(b).

The kit for forming the injectable composition contains a first component (a), and a second component (b) capable of forming a three-dimensional matrix, clot or gel when combined together. The kit also contains a third component (c) in particular in a third container, which contains a strontium salt. The kits may also contain instructions for combining the different components as well as one or more devices for mixing and/or applying the components such as syringes, pipettes, pipette bulbs, vials, and the like. In one embodiment, the kit is in the form of a two-way syringe device. The components, active agent(s), carriers, excipients, etc. are mixed by squeezing the contents of both syringes through a mixing chamber and/or needle and/or static mixer. The components (a) and (b), herein referred to as “the precursor components”, may be in the form of a solid, such as a dry powder or may in solution, such as in a buffer. If the precursor components are in the form of a solid, the kit may contain buffer solutions and instructions for preparing solutions of the precursor components. The strontium salt is provided either in a separate container, which is attachable to the two-way syringe and the strontium salt is in the form of a powder; or it can be in solution with the fibrinogen or thrombin components.

In one embodiment, the first container (a) in the kit contains fibrinogen and the second precursor container (b) contains thrombin which, when combined together, form a fibrin matrix. Fibrinogen is dissolved (optionally with aprotinin to increase stability) in a buffer solution at physiological pH (in a range from pH 6.5 to 8.0, preferably from pH 7.0 to 7.5) and is stored separately from a solution of thrombin in a calcium chloride buffer (e.g. concentration range of from 40 to 50 mM). The buffer solution for the fibrinogen can be a histidine buffer solution at a preferred concentration of 50 mM including additionally NaCl at a preferred concentration of 150 mM or TRIS buffer saline (preferably at a concentration of 33 mM).

In a preferred embodiment, both fibrinogen and thrombin are stored separately from each other in lyophilized form. Prior to use, a tris or histidine buffer is added to the fibrinogen, and the thrombin is dissolved in a calcium chloride solution. Subsequently, the fibrinogen and the thrombin solutions are placed in separate containers/vials/syringe bodies and mixed by a two way connecting device, such as a two-way syringe. Optionally, the containers/vials/syringe bodies are bipartite devices, having two chambers separated by an adjustable partition which is perpendicular to the syringe body wall. One of the chambers contains the lyophilized fibrinogen or thrombin, while the other chamber contains an appropriate buffer solution. When the plunger is pressed down, the partition moves and releases the buffer into the fibrinogen chamber to dissolve the fibrinogen. Once both fibrinogen and thrombin are dissolved, both bipartite syringe bodies are attached to a two-way connecting device and the contents are mixed by squeezing them through the injection needle attached to the connecting device. Optionally, the connecting device contains a static mixer to improve mixing of the contents.

In another embodiment, containers (a), (b) or (c) may further contain a parathyroid hormone. Preferably the parathyroid hormone is TGplPTH1-34 and even more preferably, the PTH is included in the first container (a).

In another embodiment, the kit may comprise a fourth container comprising a dry, powder iodinated contrast agent or an iodinated contrast agent in solution.

### IV. Methods of Making Injectable Compositions

The injectable tissue augmentation compositions are prepared by first mixing component (c) with either components (a), or component (b) and subsequently by mixing the mixture of components (c) and (a) with component (b) or by mixing the mixture of components (c) and (b) with component (a). Alternatively, the injectable tissue compositions can be prepared by mixing component (c) with the mixture of components (a) and (b). Preferably a homogenous mixture is formed. For example, a fibrinogen solution can be transferred into a 10 cc luer-lock syringe, the thrombin solution can be transferred into a 10 cc luer-lock syringe and the strontium salt in a powder form can be transferred into a 10 cc luer-lock syringe.

The fibrinogen solution can be prepared in an appropriate buffer, for example histidine buffer solution at a preferred concentration of 50 mM including additionally NaCl at a preferred concentration of 150 mM or TRIS buffer saline (preferably at a concentration of 33 mM). The solution is homogenized and centrifuged to remove bubbles and sterilized, such as by filtering through a 0.22 μm filter.

The thrombin solution can be prepared in a thrombin dilution buffer, for example a buffer containing about 40 mM CaCl₂ in double-distilled water. The solution is homogenized and centrifuged to remove bubbles and sterilized, such as by filtering through a 0.22 μm filter.

The strontium salt is sterilized by irradiation or any other suitable method for powder sterilization.

Optionally, the first component may further comprise parathyroid hormone or a biologically functional fragment and preferably TGplPTH1-34.

If an iodinated contrast agent is needed, it can be stored in components (a), (b) or (c) or separately. Preferably, the iodinated contrast agent, when in solution, is stored in the thrombin component (b). When the iodinated contrast agent is in a powder form, it is stored with the strontium salt component (c) or alternatively is transferred into a 10 cc luer-lock syringe. In case the iodinated contrast agent is stored separately, it is sterilized by lyophilization (typically about 1 day at −58° C. and 0.03 mbar conditions) and subsequently ground until it reaches a visually homogenous solid powder form.

The syringes containing the fibrinogen, the thrombin, the strontium salt and optionally the iodinated contrast agent are connected through a luer lock adapter and their content is homogenized by transferring the contents from syringe to syringe thoroughly. The mixture can then be injected into a tissue.

In case, the strontium salt component is mixed to the mixture of the fibrinogen and thrombin, after the fibrinogen and thrombin solutions have been mixed together, the now empty syringe is removed and the syringe containing the strontium salt is connected via a Luer lock adapter to the fibrinogen/thrombin mixture and homogenized by transferring the contents from syringe to syringe thoroughly. Typically, the liquid fibrinogen/thrombin mixture is transferred into the strontium salt and not vice-versa to prevent the powder from clogging the syringe. Generally, the material remains liquid for approximately at least 1 minute and during this time it can be injected into the defect or alternatively after few minutes it can be delivered as a pre-formed gel.

The preparation of the injectable composition can be carried out at any suitable temperature, such as in the range from about 18 to about 37° C., for example 25° C.

In one embodiment, a method for tissue augmentation and in particular hard tissue augmentation comprises the steps of:
a) providing a first component comprising fibrinogen;
b) providing a second component comprising thrombin capable of forming a three-dimensional matrix when combined with the first precursor component of step a);
c) providing a third component comprising a strontium salt;
d) mixing the second and third components;
e) mixing the first component with the mixture of step d) to form the injectable composition;
f) injecting the injectable composition into the tissue in need of augmentation to form a three-dimensional matrix;

In a further embodiment, the tissue of step f) is trabecular hard tissue.

### V. Uses for the Injectable Compositions

The injectable compositions described herein can be injected into the site of need, after which the compositions clot or form a fibrin matrix. They can be used to reduce, treat, or prevent fractures or tears in tissues and in particular in hard tissues. The composition can be used in vertebroplasty, kyphoplasty, bone repair, disc repair, tendon repair, ligament repair, or cartilage repair. Generally, the compositions are injected into a space in the tissue, for example into trabecular bone. Once injected into the porous area of a tissue, the composition forms a clot or matrix which strengthens the tissue.

One embodiment provides a method for treating or preventing a tissue disease by injecting the disclosed compositions into a porous region of the tissue and allowing the composition to form a gel, clot, or crosslinked matrix. Representative hard tissue disease includes disease of the bone, teeth, cartilage, disc or vertebra and tendon or ligament, including, but not limited to, osteoporosis.

The compositions can be used as a prophylactic agent to prevent or reduce the risk of fracture in a hard tissue by injecting the hard tissue with the compositions.

The composition initially strengthens the tissue followed by a regenerating effect of the tissue through degradation and release of active agents, such as small and/or large molecules, peptides, or proteins. In a preferred embodiment, a PTH preferably PTH1-34 is included in the composition.

### EXAMPLES

### Example 1

### Viscosity of Various Fibrin Compositions Over Time

### Materials

Fibrin sealant solution: Fibrinogen sealant solution at concentration of 80 mg/ml.

Thrombin 510 IU/ml: Thrombin at 510 IU/ml in solution.

Thrombin buffer: 40 Mm CaCl₂ in H₂O.

Iodixanol: 5-(acetyl-(2,3-dihydroxypropyl)amino)-N,N′-bis(2-3-dihydroxypropyl)-2,4,6-triiodo-benzene-1,3-dicarboxamide.

Iohexyl: 1,3-benzene-dicarboxamide, 5-[acetyl (2,3-dihydroxypropyl)amino]-N,N′-bis(2,3 dihydroxyproplyl)-2,4,6-triiodo.

Iopamidol: (S)—N,N′-bis[2-dihydroxy-1-(hydroxymethyl)ethyl]-5-[(2-hydroxy-1-oxopropyl)amino]-2,4,6,-triiodo-1,3-benzenedicarboxamide.

Tricalcium phosphate: 10 micron spherical (Biotal).

A 391 U/ml thrombin solution is prepared in a thrombin dilution buffer (40 Mm CaCl₂ in double distilled water). The solution is homogenized and centrifuged to remove bubbles and sterilized by filtering through a 0.22 μm filter. The contrast agent is sterilized by lyophilization (typically about 1 day at −58° C. and 0.03 mbar conditions) and subsequently grinded until it reaches a visually homogenous solid powder form.

The sterile fibrinogen (a) (at 80 mg/ml) is mixed with the thrombin (b) in a 2.5:1 volume/volume ratio such that the Volume (a)+(b) comprises about 57 mg/ml fibrinogen and about 11 IU/ml thrombin.

2.5 cc fibrinogen solution at 80 mg/ml are transferred into a 10 cc luer-lock syringe.

1 cc thrombin solution at 391 U/ml is transferred into a 10 cc luer-lock syringe.

1.4 g of Iopamidol powder are weighed in a 10 cc luer-lock syringe (400 mg powder per ml of fibrin).

The syringes containing the fibrinogen and the thrombin are connected through a luer lock adapter and their content is homogenized by transferring the contents from syringe to syringe thoroughly.

The syringe containing the fibrinogen/thrombin mixture and the syringe containing the contrast agent powder are connected via a Luer lock adapter and their content is homogenized by transferring the contents from syringe to syringe thoroughly. For ease of handling, first the liquid fibrinogen/thrombin mixture is transferred into the contrast agent powder and not vice-versa. The material remains liquid for approximately 1 minute and during this time it can be injected into the defect or alternatively after few minutes it can be delivered as a pre-formed gel.

The following compositions were prepared: (F1) Fibrinogen 57 mg/ml of fibrin, Thrombin 11 IU/ml of fibrin, 400 mg Iopamidol powder per ml of fibrin, 0.1 g 10 micron tricalcium phosphate powder per ml fibrin; (F2) Fibrinogen 57 mg/ml of fibrin, Thrombin 11 IU/ml of fibrin, 400 mg Iopamidol powder per ml of fibrin; (F3) Fibrinogen 40 mg/ml of fibrin, Thrombin 11 IU/ml of fibrin, 400 mg Iopamidol powder per ml of fibrin; (F4) Fibrinogen 57 mg/ml of fibrin, Thrombin 11 IU/ml of fibrin, 400 mg Iohexyl powder per ml of fibrin; (F5) Fibrinogen 40 mg/ml of fibrin, Thrombin 11 IU/ml of fibrin, 400 mg Iopamidol in solution together in the thrombin syringe; (F6)—Fibrinogen 42 mg/ml of fibrin, thrombin 11 IU/ml of fibrin, and 400 mg Iopamidol in solution per ml/fibrin; and (F7) Fibrinogen 42 mg/ml of fibrin, thrombin 4 IU/ml of fibrin, 250 mg Iodixanol per ml of fibrin (in solution in thrombin dilution buffer, separated from thrombin before mixing), and 15 mg/ml PEG 4 arms —OH terminated, MW=10,000 Da

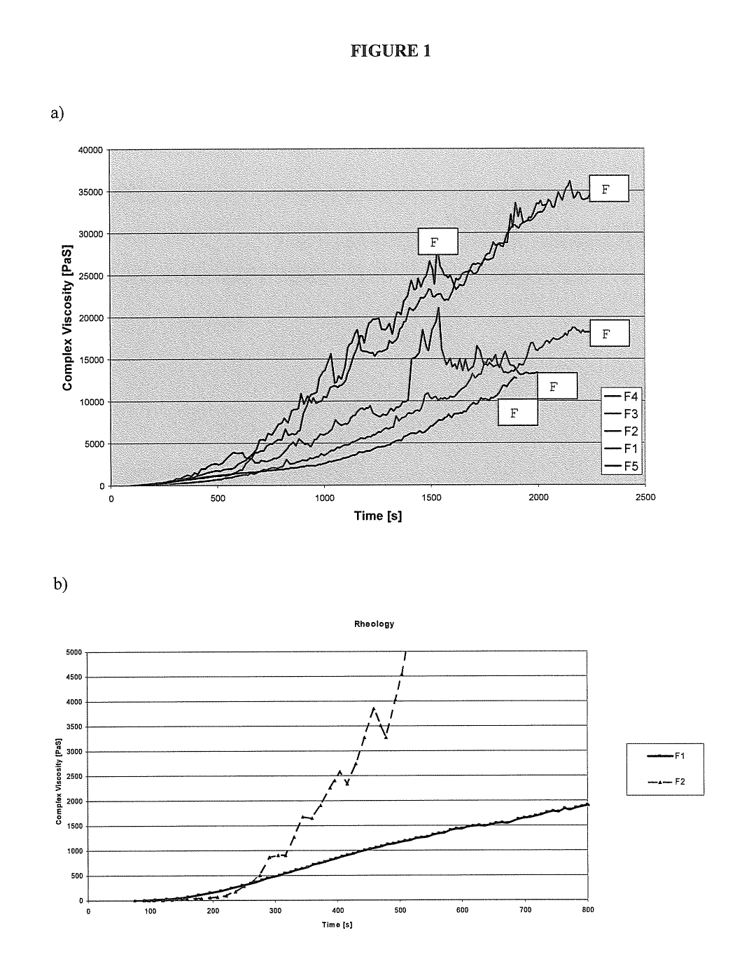

The viscosity over time of respective clots with different contrast agents and fibrinogen concentrations is shown in FIG. 1a. In particular, the graph shows: a higher viscosity is provided by the mixing of iodine in powder versus liquid form (Composition F3 vs F5); a higher viscosity is provided by the increase of fibrinogen concentration (F3 vs F2); a higher viscosity is provided by Iopamidol versus Iohexyl (F2 vs F4); and a similar viscosity is provided by two formulations with and without the incorporation of calcium salts (tricalcium phosphate 10 micron powder).

As shown in FIG. 1b, the addition of PEG improves the viscosity of the fibrin clots containing iodinated contrast agent. The graph shows that higher viscosity is provided by the mixing of iodinated contrast agent and PEG as compared to compositions containing iodinated contrast agent alone (no PEG).

### Example 2

### Preparation of Clots Containing PEG

### Materials

Fibrin sealant solution: Fibrinogen sealant solution at concentration of 84 mg/ml buffer solution

Thrombin 556 IU/ml: Thrombin at 556 IU/ml in solution.

Thrombin buffer: 40 Mm CaCl2 in H2O.

PEG: 4 arms —OH terminated, MW=10,000 Da

### Preparation of Precursor Components

Fibrinogen was provided sterile.

A solution of thrombin and iodinated contrast agent were prepared in a thrombin dilution buffer (40 Mm CaCl₂ in double distilled water). When applicable, PEG was also dissolved in the solution. The solution was homogenized and centrifuged to remove bubbles and sterilized by filtering through a 0.22 μm filter.

Filler was sterilized by gamma or beta irradiation.

Components were filled in 10 ml luer lock syringes.

### Formation of Injectable Compositions

Six (6) injectable compositions (labeled herein as “C1”-“C6”, respectively) were prepared. Concentrations listed below are per ml of the combined thrombin and fibrinogen solutions including any compounds dissolved therein, such as PEG, but excluding any different powdered components if they were added separately.

“PEG” as used in this example, refers to a 4-armed PEG with a MW of 10,000 Da, wherein all of the arms are —OH terminated.

C1: Fibrinogen 42 mg/ml, thrombin 37.5 IU/ml, Iodixanol in solution together with thrombin before mixing at 400 mg/ml. No PEG.

Kits were prepared as follows: 2 cc fibrinogen+2 cc thrombin (75 IU/ml)

C2: Fibrinogen 42 mg/ml, thrombin 3.6 IU/ml, PEG 18 mg/ml, 400 mg/ml Iodixanol.

Kits were prepared as follows: 2.5 cc fibrinogen+0.5 cc thrombin (20 IU/ml)+0.1 g PEG and 2 g Iodixanol dissolved in 2 cc thrombin dilution buffer

C3: Fibrinogen 42 mg/ml, thrombin 41 U/ml, Iodixanol in solution separated from thrombin before mixing at 250 mg/ml, 15 mg/ml PEG.

Kits were prepared as follows: 2 cc fibrinogen+0.5 cc thrombin (321 U/ml)+1.5 cc [666 mg/ml Iodixanol+0.06 g PEG]

C4: Fibrinogen 42 mg/ml, thrombin 41 U/ml, Iodixanol in solution separated from thrombin before mixing at 250 mg/ml, 11.2 mg/ml PEG.

Kits were prepared as follows: 2 cc fibrinogen+0.5 cc thrombin (321 U/ml)+1.5 cc [666 mg/ml Iodixanol+0.045 g PEG]

C5: Fibrinogen 42 mg/ml, thrombin 41 U/ml, Iodixanol in solution separated from thrombin before mixing at 250 mg/ml, 18.7 mg/ml PEG.

Kits were prepared as follows: 2 cc fibrinogen+0.5 cc thrombin (321 U/ml)+1.5 cc [666 mg/ml Iodixanol+0.075 g PEG]

C6: Fibrinogen 32 mg/ml, thrombin 41 U/ml, Iodixanol in solution separated from thrombin before mixing at 250 mg/ml, 17 mg/ml PEG.

Kits were prepared as follows: lcc fibrinogen+0.5 cc thrombin (241 U/ml)+1.13 cc [666 mg/ml Iodixanol+0.045 g PEG 4 arms 10K]

Formulations without PEG (e.g. C1) produced very brittle matrices that break easily when compressed between two fingers.

Formulations containing PEG produced tougher matrices, which required the application of higher compressive strength to be broken and showed much higher resistance in tension than C1 (pulling with fingers). These results also indicate the difference between fibrous and amorphous structure of compositions with and without PEG respectively.

Similar results to those obtained with 4-armed, hydroxyl terminated PEG, having a molecular weight of 10,000 Da were obtained with the following PEGs at similar concentrations to those used in composition C1 to C6: PEG linear —OH terminated MW=1,000 Da, MW=6,000 Da, MW=10,000 Da, MW=20,000 Da.

### Clot Turbidity

The formation of a fibrous structure in presence of PEG was confirmed by visual inspection of the compositions during clotting.

Formulations without PEG (C1) remained transparent, while those containing PEG became opaque, thus indicating the formation of a fibrous structure.

The higher the concentration of PEG, the higher the turbidity of the clot.

For high concentrations of PEG, e.g. more than the double of the concentrations for each of formulations C1 to C6, aggregates of fibers started forming in the clot immediately after the mixing procedure was complete. Such aggregates eventually clotted together, but a phase separation between the fibrin clot and residual buffer was observed. Such a formulation is less suitable for hard tissue augmentation than one that does not show phase separation.

The optimal concentration of PEG depends on the amount of contrast agent in the clot.

Similar results to those obtained with 4-armed, hydroxyl terminated PEG, having a molecular weight of 10,000 Da were obtained with the following PEGs at similar concentrations to those used in compositions C1 to C6: PEG linear —OH terminated MW=1,000 Da, MW=6,000 Da, MW=10,000 Da, MW=20,000 Da.

### Example 3

### Mechanical Properties

### Extrusion Energy

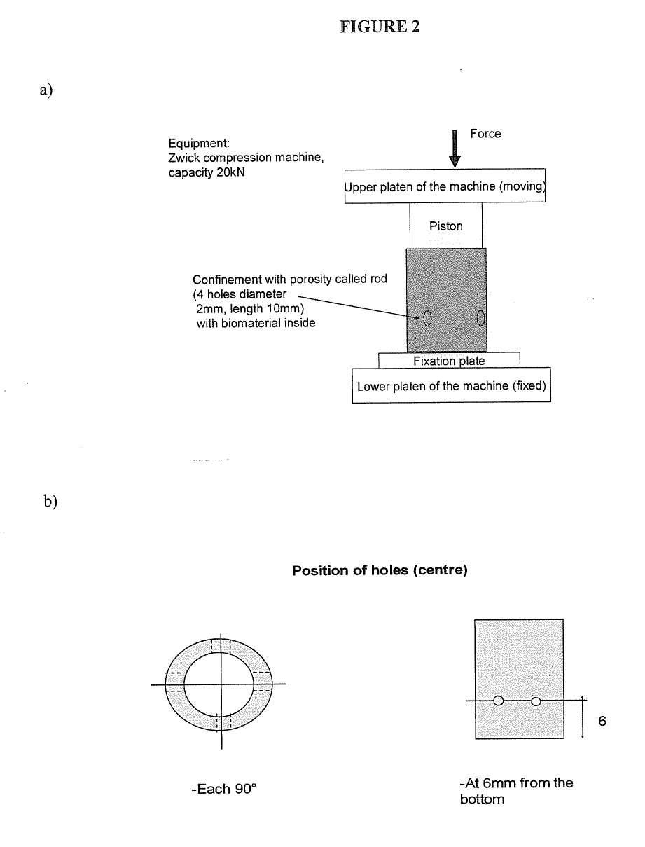

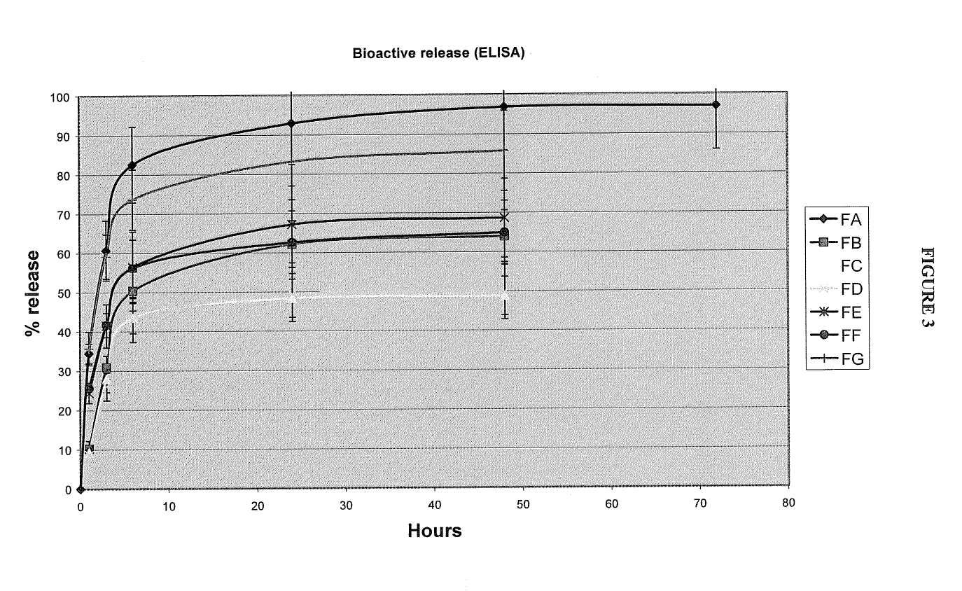

Clots containing iodinated contrast agent are prepared as described in Example 1. Immediately after completion of the mixing procedure, 4 cc of the composition are injected when still liquid in a stainless steel cylinder, where four 2 mm diameter holes are made at 6 mm from the base (see FIG. 2). A stainless steel piston is then placed into the cylinder and is compressed with a MTS machine (control in displacement), thus extruding the composition out of the holes. The compression load was measured throughout the test. A load-displacement graph was prepared based on these measurements. The energy needed for the extrusion of the composition was calculated as the area under the load/displacement curve.

This model represents the in vivo injection into a vertebra, where the holes in the cylinder mimic blood vessels and represent the preferred path for leakage. In particular, the model provides an idea on the potential risk of leakage associated with the tested material. The higher the extrusion energy, the lower the in vivo risk of leakage in a vertebra.

Table 3 shows the advantage of adding the contrast agent in powder rather than liquid form, and of increasing the concentration of fibrinogen, as represented by Ff, Fg and Fh. Moreover, the extrusion energy increases in general over time, as for example represented by Fb and Fd (same composition at different time point). The extrusion energy is also higher for formulations with higher thrombin concentration, as for example represented by Fg and Fe. In general, the presence of inorganic salt contrast agents significantly increases the extrusion energy, as represented by Fl, Fm and Fn. The composition containing PEG (Fo) required a significantly greater amount of energy to extrude the composition than the composition formed in the absence of PEG. Thus the composition containing PEG would be expected to have a the lower the in vivo risk of leakage in a vertebra than the composition without PEG.

TABLE 3

| | | Time elapsed after completion | Extrusion |
|---|---|---|---|
| | Composition | of mixing | energy |
| Fa | F = 41 mg/ml of fibrin | 20 min | 360 mJ |
| | T = 13 IU/ml of fibrin | | |
| | 359 mg Iopamidol in solution per ml/fibrin | | |
| Fb | F = 53 mg/ml of fibrin | 20 min | 658 mJ |
| | T = 11 IU/ml of fibrin | | |
| | Iopamidol 400 mg powder per ml/fibrin | | |
| Fc | F = 60 mg/ml of fibrin | 20 min | 916 mJ |
| | T = 11 IU/ml of fibrin | | |
| | Iopamidol 400 mg powder per ml/fibrin | | |
| Fd | F = 53 mg/ml of fibrin | 35 min | 857 mJ |
| | T = 11 IU/ml of fibrin | | |
| | Iopamidol 400 mg powder per ml/fibrin | | |
| Fe | F = 60 mg/ml of fibrin | 10 min | 1900 mJ |
| | T = 7.6 IU/ml of fibrin | | |
| Ff | F = 42 mg/ml of fibrin | 20 min | 558 mJ |
| | T = 2.0 IU/ml of fibrin | | |
| Fg | F = 60 mg/ml of fibrin | 20 min | 1015 mJ |
| | T = 2.0 IU/ml of fibrin | | |
| Fh | F = 72 mg/ml of fibrin | 20 min | 1283 mJ |
| | T = 2.0 IU/ml of fibrin | | |
| Fi | F = 60 mg/ml of fibrin | 20 min | 670 mJ |
| | T = 7.0 IU/ml of fibrin | | |
| | Iodixanol 450 mg/ml in powder | | |
| Fl | F = 56 mg/ml of fibrin | 20 min | 3763 mJ |
| | T = 2.0 IU/ml of fibrin | | |
| | Strontium carbonate 40% w/v | | |
| Fm | F = 42 mg/ml of fibrin | 20 min | 2439 mJ |
| | T = 4.0 IU/ml of fibrin | | |
| | Iodixanol 150 mg/ml in solution | | |
| | Strontium carbonate 25% w/v | | |
| Fn | F = 42 mg/ml of fibrin | 20 min | 1872 mJ |
| | T = 12.5 IU/ml of fibrin | | |
| | Iodixanol 400 mg/ml in solution | | |
| | Tri-calcium phosphate 25% w/v | | |
| Fo | Fibrinogen = 42 mg/ml of fibrin | 20 min | 560 mJ |
| | Thrombin = 2 IU/ml of fibrin | | |
| | 400 mg Iodixanol per ml of fibrin, in solution in thrombin | | |
| | dilution buffer, separated from thrombin before mixing | | |
| | 20 mg/ml PEG 4 arms - OH terminated, MW = 10,000 Da | | |

F = fibrinogen,
T = thrombin

Visually, clots obtained with iodinated contrast agent mixed in powder form and increased fibrinogen concentration appear more opaque than those obtained with iodinated contrast agent in liquid form. This suggests that in the first case the contrast agent has less effect on the clotting of fibrin when it is mixed in powder form. Moreover, clots obtained with higher concentrations of fibrinogen result to be much more resistant in tension and tear than those with lower concentration of fibrinogen.

Clots obtained with higher concentrations of fibrinogen and no iodine show excellent resistance and elasticity in tension, such that they can be strongly pulled with two hands without breaking and they can be knotted.

### Compressive Stiffness

Clots containing various concentrations of fibrinogen, with and without iodinated contrast agents, are prepared as described in Example 1 starting from an initial fibrinogen concentration of 82 or 84 mg/ml and an initial thrombin concentration of 518 IU/ml. For formulations comprising strontium carbonate (CAS number 1633-05-2, Sigma-Aldrich), the latter component is present in powder form and is filled in a separate syringe, similar to the procedure followed for the preparation of the iodine powder. 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 60 mg/ml of fibrin, Thrombin 7 IU/ml of fibrin, Iodixanol 450 mg/ml of fibrin in powder form; 5) Fibrinogen 56 mg/ml of fibrin, Thrombin 2 IU/ml of fibrin, strontium carbonate 40% w/v powder (added to the mixture after the fibrinogen and thrombin components have been mixed), 6) Fibrinogen 42 mg/ml, thrombin 41 U/ml, iodixanol 150 mg/ml pre-dissolved in the thrombin solution, strontium carbonate 25% w/v in powder, 7) Fibrinogen 42 mg/ml, thrombin 21 U/ml, iodixanol 400 mg/ml pre-dissolved in the thrombin solution, tri-calcium phosphate 25% w/v in powder.

Clots are prepared in comparable volumes and immediately after mixing moulded in cylindrical specimens of 12 mm diameter and 12 mm height. Clots are kept in the mould for 20 minutes before extraction. Afterwards, an uniaxial compressive load at a constant strain rate is applied under the following conditions:

| | |
|---|---|
| preload: | 0.025 N |
| interval between reaching preload and test start: | 5 s |
| strain rate: | 5 mm/min. |
| test end: | 75% nominal strain |

At completion of the compressive test, the load is released and the following parameters are recorded:
Initial elastic modulus (by linear interpolation of the stress-strain curve between 1 and 2% of strain);
Deformation energy until 50% of strain

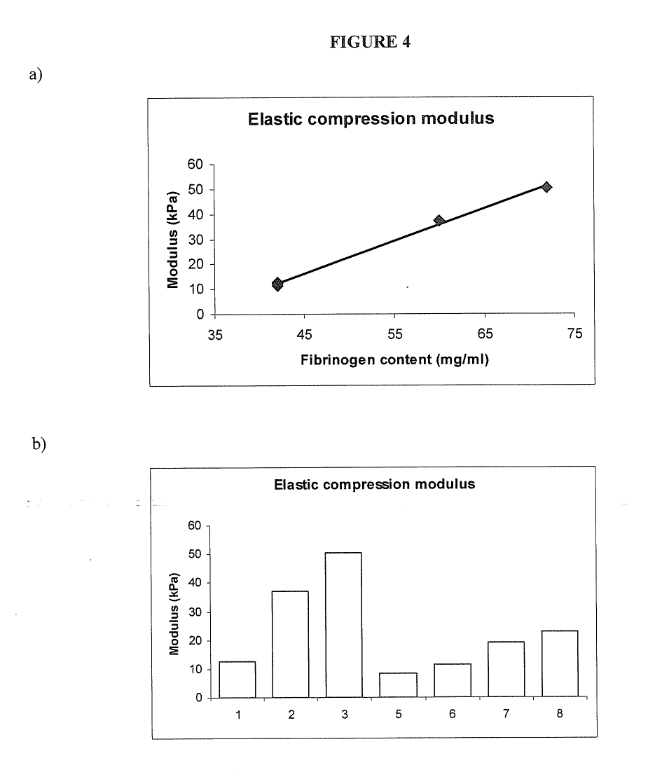

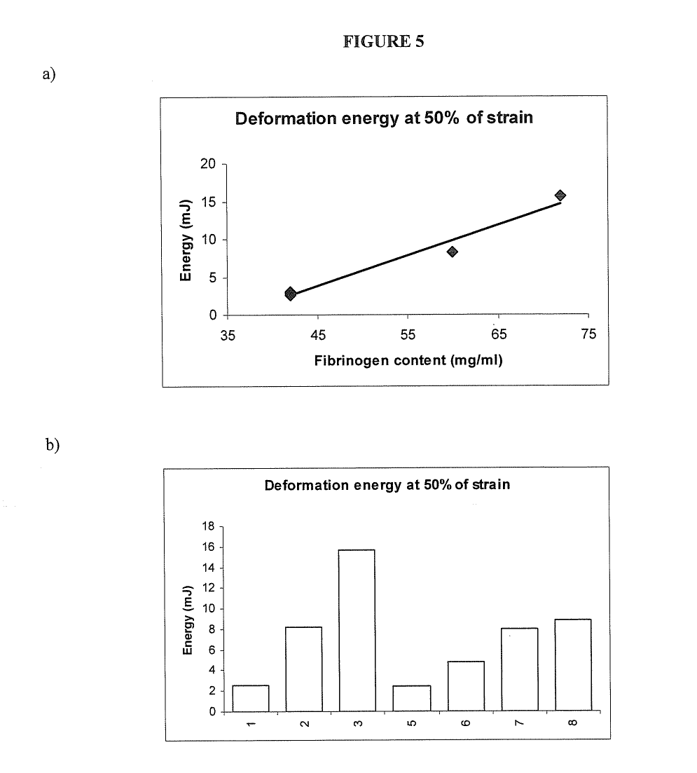

The comparison between formulations with the same amount of thrombin and increasing concentration of fibrinogen (number. 1, 2 and 3) is presented in FIG. 4 and FIG. 5 where it is shown that higher concentration of fibrinogen leads to higher elastic modulus (FIG. 4a) and deformation energy (FIG. 5a). Data for all formulations 1) to 7) are reported in FIG. 4b and FIG. 5b respectively, where it is shown that the incorporation of inorganic salt contrast produce in general an increase in the elastic modulus and deformation energy.

### Example 4

### Suitability for Injection Under Pressure

Clots containing various concentrations of fibrinogen, with and without iodinated contrast agents, are prepared as described in Example 1 starting from an initial fibrinogen concentration of 82 or 84 mg/ml and an initial thrombin concentration of 518 IU/ml. For formulations comprising strontium carbonate (CAS number 1633-05-2, Sigma-Aldrich), the latter component is present in powder form and is filled in a separate syringe, similar to the procedure followed for the preparation of the iodine powder. 1) Fibrinogen 42 mg/ml of fibrin, Thrombin 2 IU/ml of fibrin; 2) Fibrinogen 60 mg/ml of fibrin, Thrombin 2 IU/ml of fibrin; 3) Fibrinogen 72 mg/ml of fibrin, Thrombin 2 IU/ml of fibrin; 4) Fibrinogen 84 mg/ml of fibrin, Thrombin 2 IU/ml of fibrin; 5) Fibrinogen 60 mg/ml of fibrin, Thrombin 7 IU/ml of fibrin, Iodixanol 450 mg/ml of fibrin in powder form; 6) Fibrinogen 56 mg/ml of fibrin, Thrombin 2 IU/ml of fibrin, strontium carbonate 40% w/v powder (added to the mixture after the fibrinogen and thrombin components have been mixed), 7) Fibrinogen 42 mg/ml, thrombin 41 U/ml, iodixanol 150 mg/ml predissolved in the thrombin solution, strontium carbonate 25% w/v in powder, 8) Fibrinogen 42 mg/ml, thrombin 21 U/ml, iodixanol 400 mg/ml pre-dissolved in the thrombin solution, tricalcium phosphate 25% w/v in powder

Two types of test are performed in order to detect the buffer that is potentially released after clot extrusion or compression.

Clot Extrusion with Vertebrolasty Gun

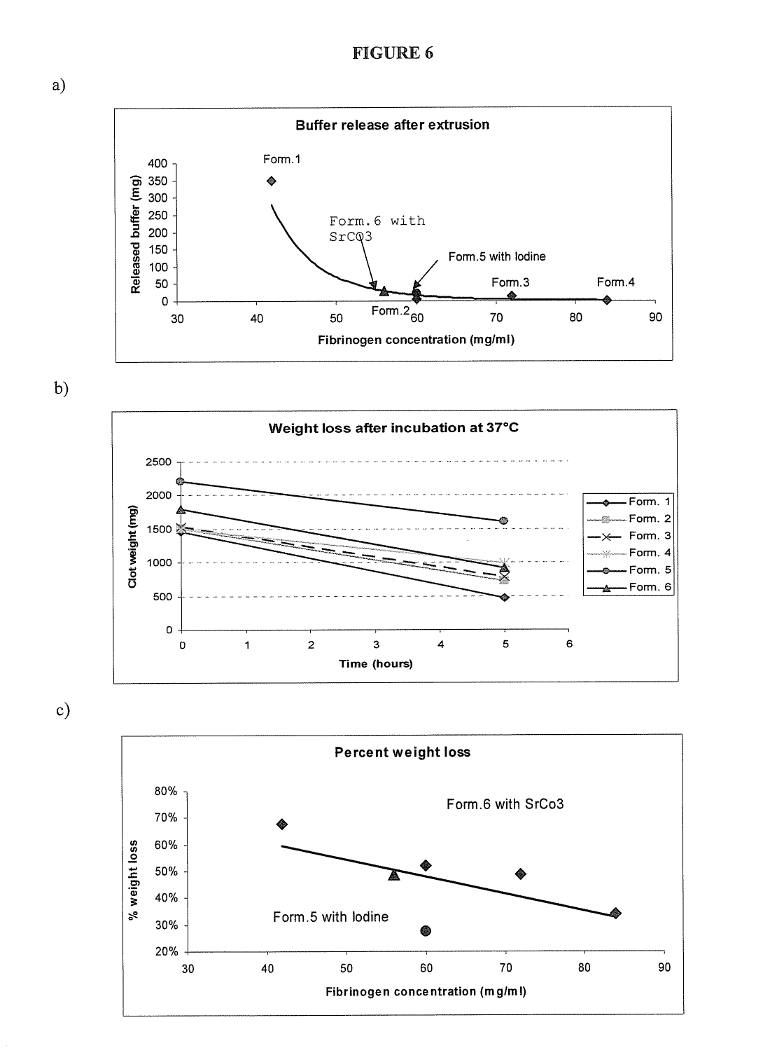

Clots from formulation 1 to 6 are prepared in comparable volumes and extruded through a 10 G 100 mm vertebroplasty trocar (Optimed) by using an Optimed Cemento-RE vertebroplasty injection gun. Due to the pressure applied during extrusion, buffer separates from the clot. Buffer is collected and the weight recorded. The extruded clot is collected in a plastic container and subsequently stored at 37° C. for 5 hours. As shown in FIG. 6a, higher concentrations of fibrinogen lead to lower buffer separation after extrusion, thus better preserving the original composition of the clot and making it more suitable for injection under pressure. This is confirmed by the lower weight loss observed for clots comprising higher fibrinogen concentrations after incubation at 37° C. for 5 hours (FIG. 6b, weight loss; FIG. 6c, % weight loss).

Clot Compression with Mechanical Testing Machine

Clots from formulation 1 to 8 are prepared in comparable volumes and immediately after mixing moulded in cylindrical specimens of diameter 12 mm and height 12 mm. Clots are kept in the mould for 20 minutes before extraction. Afterwards, the weight of the specimen is collected and an uniaxial compressive load at a constant strain rate is performed under the following condition:

| | |
|---|---|
| preload: | 0.025 N |
| interval between reaching preload and test start: | 5 s |
| strain rate: | 5 mm/min. |
| test end: | 75% nominal strain |

At completion of the compression test the load is released and the strained specimen is newly weighed. Buffer release is calculated from the difference between the weight before and after the test.

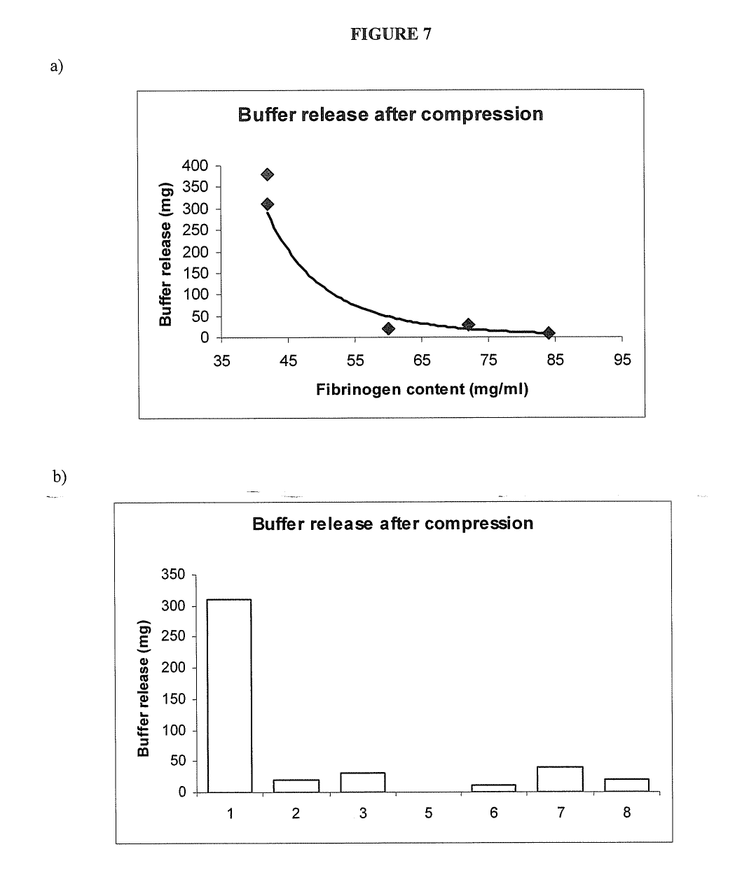

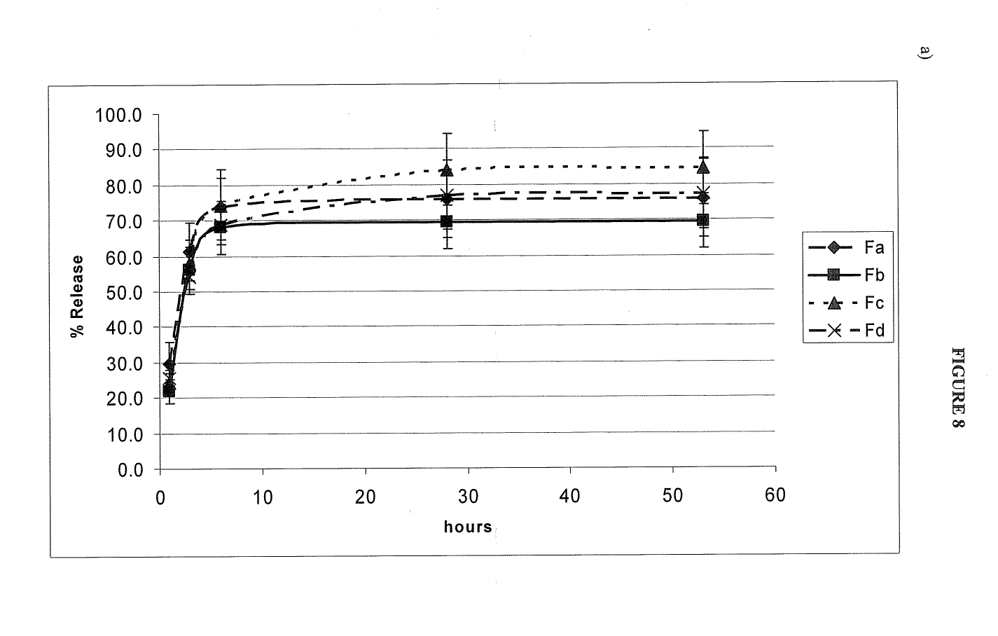

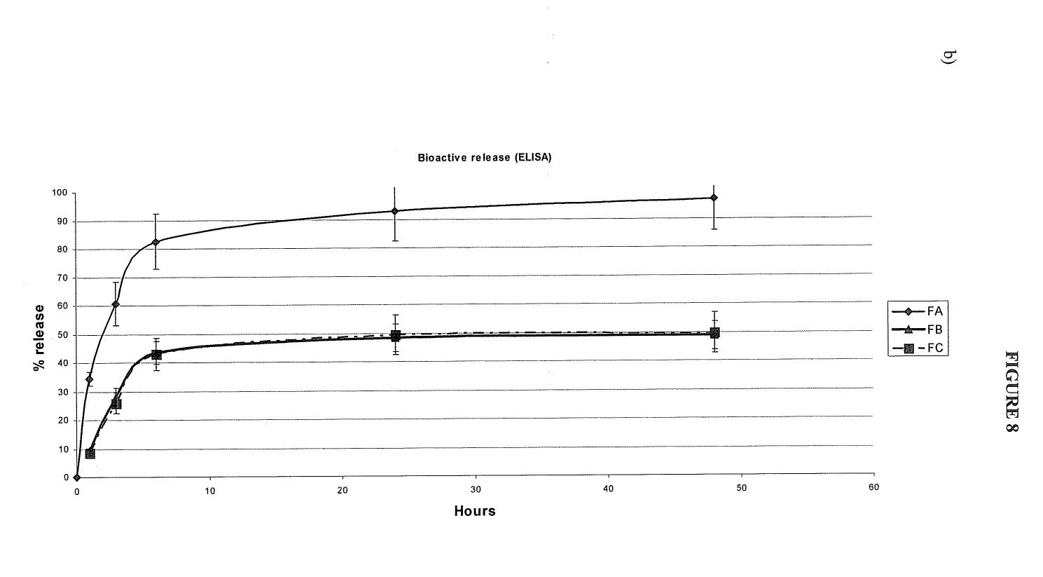

The comparison between formulations with the same amount of thrombin and different fibrinogen concentration (formulation number 1, 2, 3 and 4) is presented in FIG. 7a, where it is shown that higher concentrations of fibrinogen 60 mg/ml) lead to a drastic reduction in buffer release. Data for all formulations 1) to 8) are reported in FIG. 7b, where it is shown that the incorporation of inorganic salt contrast may also lead to a reduced buffer release.

### Example 5

### Fiber Mesh Structure

Clots containing iodinated contrast agent are prepared as described in Example 1. Clots containing strontium carbonate are prepared as described in Example 3. After 1 h incubation at 37° C. samples are prepared for scanning electron microscopy analysis (SEM).

Clots where the iodinated contrast agent is added in powder form show bigger pores than those where the contrast agent is added in liquid form and more similar to those obtained with the normal fibrin clot (no contrast agent). Clots where the iodinated contrast agent is replaced by strontium carbonate show open pore structure similar to the normal fibrin clot.

The concentration of thrombin units plays also an important role, the higher the concentration, the tighter the structure. Increasing the concentration of fibrinogen also leads to smaller pores. In general, small pores may allow lower cell infiltration, lower exchange of media between the clot and the external environment, slower degradation and potentially lower efficacy in hard tissue formation. It is therefore important to find the right compromise between pore size and clot strength.

An indication of the structure of the clot is also given by the release test when a bioactive is physically incorporated in the composition. The following compositions were compared: (FA) fibrinogen 45 mg per ml of fibrin, thrombin 2 IU per ml of fibrin; (FB) fibrinogen 54 mg per ml of fibrin, thrombin 4 IU per ml of fibrin, 300 mg Iodixanol per ml fibrin (Iodixanol in solution stored separately from fibrinogen and thrombin and added to fibrinogen/thrombin mixture once the two have been mixed together); (FC) fibrinogen 45 mg per ml of fibrin, thrombin 37.5 IU per ml of fibrin, 300 mg Iodixanol per ml fibrin (Iodixanol in solution together with thrombin component); (FD) fibrinogen 45 mg per ml of fibrin, thrombin 37.5 IU per ml of fibrin, 400 mg Iodixanol per ml fibrin (Iodixanol in solution together with thrombin component); (FE) fibrinogen 44 mg per ml of fibrin, thrombin 11 IU per ml of fibrin, 303 mg Iodixanol per ml fibrin (Iodixanol in solution stored separately from fibrinogen and thrombin and added to fibrinogen/thrombin mixture once the two have been mixed together); (FF) fibrinogen 48 mg per ml of fibrin, thrombin 4 IU per ml of fibrin, 300 mg Iohexyl per ml fibrin (Iohexyl in solution stored separately from fibrinogen and thrombin and added to fibrinogen/thrombin mixture once the two have been mixed together); and (FG) fibrinogen 53 mg per ml of fibrin, thrombin 10 IU per ml of fibrin, 400 mg Iopamidol in powder per ml fibrin. FIG. 3 shows that almost 100% of the bioactive is released within 48 h from a normal fibrin clot (2 IU/ml thrombin, no iodine). When the contrast agent is present in liquid form together with the thrombin component, the amount of bioactive detected at 48 h is about 50%; when the contrast agent is in solution but is stored separately from fibrinogen and thrombin and is added to the composition only after fibrinogen and thrombin have been mixed, the release at 48 h is about 65%; when the contrast agent is mixed in powder form the detected bioactive amount is close to 90% and the release profile is similar to the one of the normal fibrin clot. This indicates that mixing the iodinated contrast agent in powder form may alter the fibrin structure much less than when it is mixed in liquid form.

The fact that the clotting process is less influenced by the presence of iodinated contrast agent in powder versus liquid form is supported by the analysis of the clotting times. At the same amount of thrombin units it takes much longer to clot when the iodine is liquid. Alternatively, the units of thrombin can be drastically lowered for compositions including the iodine in powder versus liquid form in order to obtain comparable clotting times (see Table 4).

TABLE 4

| | | | | Gellation |
|---|---|---|---|---|
| Fibrinogen | Thrombin | Iodine | Filler | time |
| 40 mg/ml | 2 IU/ml | NA | NA | ~1 min |
| 40 mg/ml | 2 IU/ml | 400 mg/ml Iodixanol in solution with | NA | ~45 min |
| | | thrombin | | |
| 40 mg/ml | 2 IU/ml | 400 mg/ml | 36% TCP | ~45 min |
| | | Iodixanol in solution with thrombin | 10um | |
| 40 mg/ml | 2 IU/ml | 400 mg Iodixanol powder/ml fibrin | NA | ~10 min |
| 53 mg per ml of | 11 IU per ml of | 400 mg Iopamidol in powder per ml | NA | ~1 min 10 s |
| fibrin | fibrin | fibrin | | |
| 64 mg per ml of | 10 IU per ml of | 400 mg Iopamidol in powder per ml | NA | ~1 min 10 s |
| fibrin | fibrin | fibrin | | |
| 60 mg/ml of fibrin | 7.6 IU/ml fibrin | NA | NA | ~40 s |

### Example 6

### Application of the Injectable Hard Tissue Augmentation Composition

Osteoporosis and age related loss in hard tissue mineral density often leads to fractures in vertebra. Injection of the injectable hard tissue augmentation composition according to the present invention at these sites helps in the treatment of such injuries and can also be used prophylactically to prevent injuries. Under fluoroscopy (C-arm) a vertebroplasty trocar (diameter: 3.2 mm (10 G), length 100 mm) is placed in a vertebra of a human cadaver using transpedicular approach.

A composition containing fibrinogen 53 mg per ml fibrin, thrombin 11 IU per ml fibrin, Iopamidol 400 mg powder per ml of fibrin is prepared as described in Example 1. Once the mixing procedure is completed, the liquid material is transferred into a vertebroplasty gun (Optimed Cemento-RE). The gun is directly connected to the trocar (without a tube in between) and the material is applied to the vertebra by screwing it out of the gun. The injection was performed at 34 min after completion of the mixing procedure. About 6 cc of material were injected in T9 without any leakage (data not shown).

Under fluoroscopy (C-arm) two vertebroplasty trocars (diameter: 3.2 mm (10 G), length 100 mm) are placed in a vertebra of a pig cadaver using transpedicular approach. A composition containing fibrinogen 41 mg/ml, thrombin 21 U/ml, Iodixanol 150 mg/ml in solution in the thrombin syringe, strontium carbonate 25% w/v is prepared as described in Example 1 and Example 3. First, the fibrinogen component is mixed with the iodine containing thrombin component; then, the mixture is homogenized with strontium carbonate. Once the mixing procedure is completed, the liquid material is transferred into a vertebroplasty gun (Optimed Cemento-RE). 15 min after completion of the mixing procedure the material was injected into T11. The material showed to possess suitable properties for injection into a pig vertebra.

### Example 7

### Release of Bioactive Agent

Compositions containing the components listed below were prepared according to the method described in Example 1.

The following compositions (labeled “Fa”-“Fd”), including a bioactive (same concentration for each composition), were tested (ELISA):

(Fa)—42 mg/ml fibrinogen and 2 IU/ml thrombin (normal fibrin clot, no contrast agent, no PEG);
(Fb)—42 mg/ml fibrinogen, 2 IU/ml thrombin, 400 mg/ml Iodixanol (in solution stored separately from fibrinogen and thrombin and added to fibrinogen/thrombin mixture once the two were mixed together), and 20 mg/ml PEG 4 arms —OH terminated, MW=10,000 Da
(Fc)—42 mg/ml fibrinogen, 2 IU/ml thrombin, 250 mg/ml Iodixanol (in solution stored separately from fibrinogen and thrombin and added to fibrinogen/thrombin mixture once the two were mixed together), and 12.5 mg/ml PEG 4 arms —OH terminated, MW=10000
(Fd)—42 mg/ml fibrinogen, 2 IU/ml thrombin, 250 mg/ml Iodixanol (in solution stored separately from fibrinogen and thrombin and added to fibrinogen/thrombin mixture once the two were mixed together), 12.5 mg/ml PEG 4 arms —OH terminated, MW=10000, and 15% w/v SrCO₃.

The addition of 15% w/v strontium carbonate in combination with 250 mg/ml Iodixanol, confers contrast similar to compositions containing 400 mg/ml Iodixanol.

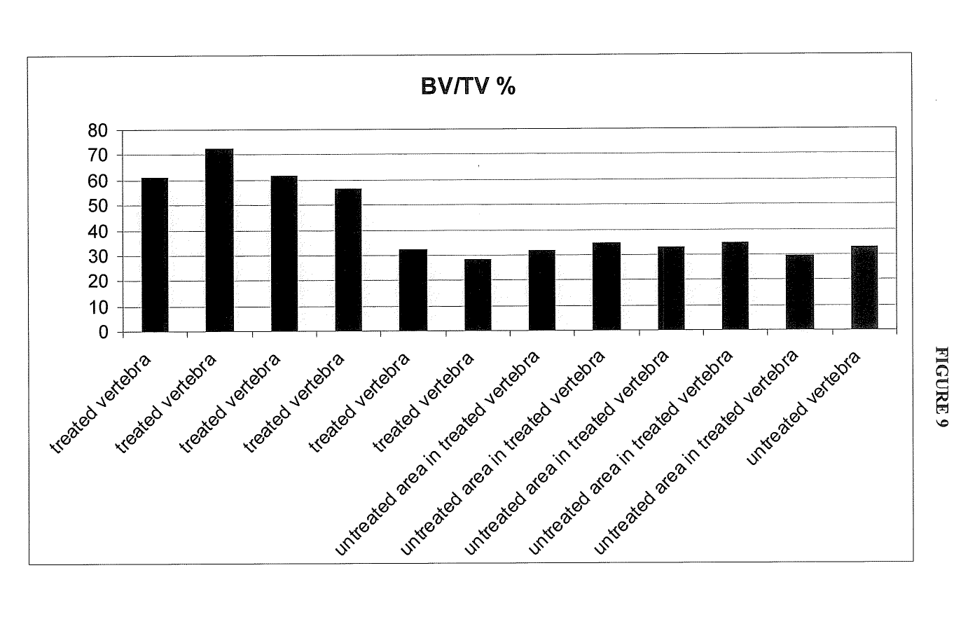

The results showing % release of the bioactive from each of the compositions listed above over time (60 hours) are provided in FIG. 8a. As shown in FIG. 8a, the release of a bioactive from compositions containing PEG was comparable to the release of a normal fibrin clot (no PEG, no contrast agent), indicating a similar clot structure.

The same release test was performed separately on compositions not including PEG. The normal fibrin clot (Fa) was used again as a reference. The following compositions (labeled “Fa”-“Fc”) were tested:

(Fa)—45 mg/ml fibrinogen and 2 IU/ml thrombin (Osteogenic Gel);
(Fb) 45 mg/ml fibrinogen, 37.5 IU/ml thrombin, and 300 mg/ml Iodixanol (in solution together with thrombin); and
(Fc) 45 mg/ml fibrinogen, 37.5 IU/ml thrombin, and 400 mg/ml Iodixanol (in solution together with thrombin).

The results showing % release of the bioactive from each of the compositions listed above over time (60 hours) are provided in FIG. 8b. As shown in FIG. 8b, the release of a bioactive from compositions containing only contrast agent (no PEG) in solution was about the half of the release of a normal fibrin clot (no PEG, no contrast agent), indicating a more compact clot structure in the compositions containing contrast agent.

### Example 8

### In Vivo Studies of the Injectable Hard Tissue Augmentation Composition in Sheep

### Vertebral Augmentation in Sheep

A 10 G vertebroplasty trocar (Optimed) is inserted transpedicularly or laterally into sheep lumbar vertebral bodies with a technique similar to the one used in vertebroplasty. The material is injected into the vertebrae with the aid of a vertebroplasty Cemento-RE gun (Optimed) and its flow is monitored by fluoroscopy. The animal is sacrificed 12 weeks post-op and new bone formation is assessed by means of microCT and histology.

The following formulations were prepared as described in Example 1:
A) Fibrinogen 60 mg/ml, thrombin 8 IU/ml, Iopamidol (powder) 425 mg/ml, TGplPTH1-34 0.4 mg/ml. Injected into the third lumbar vertebra (L3)
B) Fibrinogen 60 mg/ml, thrombin 8 IU/ml, Iodixanol (powder) 425 mg/ml, TGplPTH1-34 0.4 mg/ml. Injected into L2.
C) Fibrinogen 60 mg/ml, thrombin 8 IU/ml, Iodixanol (powder) 425 mg/ml, TGplPTH1-34 0.4 mg/ml. Injected into L5.
D) Fibrinogen 42 mg/ml, thrombin 8 IU/ml, Iodixanol (powder) 425 mg/ml, 10 micron tricalcium phosphate 15% w/v, TGplPTH1-34 0.4 mg/ml. Injected into L4.
L1 and L5 vertebrae were used as empty control (untreated).

All formulations showed adequate radio-opacity for minimally invasive application monitored by fluoroscopy. Bone volume/total volume (BV/TV) measured by microCT was in average 63% in the areas of the vertebrae where the material was injected, compared to about 30% of the untreated areas of the same treated vertebrae and untreated empty control vertebrae (FIG. 9). Results were confirmed by qualitative histological analysis.

### Femur Condyle/Proximal Tibia Augmentation in the Sheep

In this study the ability of various Vertebral Augmentation Material formulations to promote the synthesis of new bone within the inter-trabecular spacing of intact bone in the femoral condyle and proximal tibial metaphysis of the sheep was evaluated. An average volume of 2.7 mL Vertebral Augmentation Material was injected into each of the four sites per sheep (n=6 sheep per formulation)—the femoral condyle and proximal tibia on one limb with Vertebral Augmentation Material with a bioactive: TGplPTH₁₋₃₄, and the femoral condyle and proximal tibia on the contra-lateral limb with the same Vertebral Augmentation Material without the bioactive (TGplPTH₁₋₃₄). This resulted in an average TGplPTH₁₋₃₄ dose between 0.62 and 1.05 mg per bony site and between 1.25 and 2.08 mg per animal (planned dose assuming the injection of 2.7 mL material). The following formulations were tested:

1) Fibrinogen 41 mg/ml, thrombin 20 IU/ml, 10 micron tricalcium phosphate 25% w/v, Iodixanol 400 mg/ml in solution in thrombin syringe. With or without bioactive (TGplPTH₁₋₃₄).
2) Fibrinogen 59 mg/ml, thrombin 7 IU/ml, Iodixanol in powder 450 mg/ml with or without bioactive (TGplPTH₁₋₃₄).
3) Fibrinogen 41 mg/ml, thrombin 4 IU/ml, Iodixanol 150 mg/ml in solution in thrombin syringe, strontium carbonate 25% w/v, with or bioactive (TGplPTH₁₋₃₄).
4) Fibrinogen 55 mg/ml, thrombin 2 IU/ml, strontium carbonate 40% w/v, with or without bioactive (TGplPTH₁₋₃₄).
5) Fibrinogen 41 mg/ml, thrombin 2 IU/ml, with or without bioactive (TGplPTH₁₋₃₄), gold spheres 500 micron (approximately 25 spheres/ml).

Results were compared to sham controls (n=3 sheep, total of 6 sham tibias and 6 sham femurs) consisting of the needle insertion without injection of material, and to the following formulation 5) consisting in unmodified fibrin enriched with gold particles for visualization by fluoroscopy.

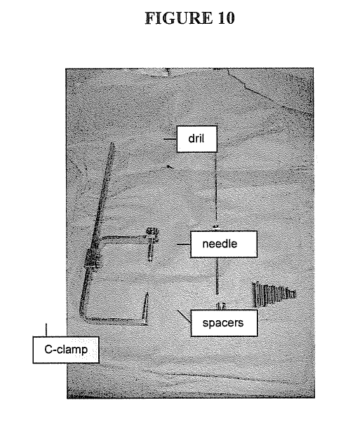

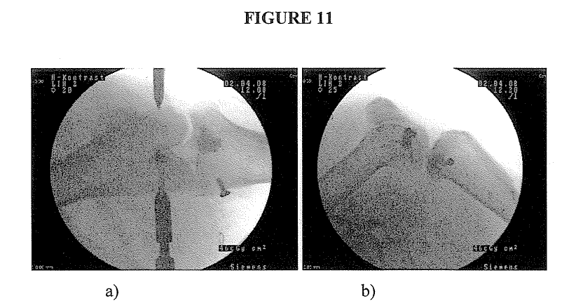

The procedure consisted of placing a 10 G (3.2 mm diameter) 100 mm-long needle with the tip ending in the trabecular portion of the femoral condyle or proximal tibial bone. This was done with the aid of a custom made aiming device (FIG. 10) that allowed exact positioning at the medial and lateral aspect of the tibia and femur including drilling in a given direction and at predictable length.

The pre-polymerized Vertebral Augmentation Material was then injected so that it was distributed within the inter-trabecular spacing of bone. After completion of the injection procedure, the needle was removed and a self-tapping screw was inserted. The screw served as a marker for identification of the insertion point and direction of the needle at sacrifice. After recovering from the anesthesia, the animals were allowed to move without restrictions.

Fluoroscopy images were taken during surgery (lateral views) and immediately post-surgery (lateral and cranio-caudal views). Radiographs were taken at 6 and 12 weeks. In vivo bone staining was performed with injections of calcein green at 6 weeks and xylenol orange two days before the 12-week sacrifice. After sacrifice, 32 mm diameter bone cylinders were cored throughout the tibia and femur by means of a diamond coated drill. The cylinders were centered at the insertion point of the needle, with the axis along the direction of the needle hole. The extracted bone cylinders were then processed for analysis by μCT, which provided the basis for true, high-resolution 3D analysis.

### Results

The surgical procedure was well tolerated. The treatment was safe up to the highest tested dose of 35 μg/kg. Intra-follow-up observations and post-mortem macroscopic examination showed no signs of toxicity for any of the Vertebral Augmentation Material formulations. One sheep had to be euthanized at 6 weeks post-operatively due to the presence of a tumor in the wall of the vagina and the urinary bladder, but unrelated to the study treatment.

Fluoroscopy images taken during and immediately post-operatively proved the suitability of all the Vertebral Augmentation Material formulations to augment the sheep femoral condyle and proximal tibial trabecular bone. In particular, the formulation containing 40% w/v strontium carbonate gave the best outcome with respect to radio-opacity, risk of leakage and handling.

Radio-opacity: all Vertebral Augmentation Material formulations showed good radio-opacity suitable for percutaneous bony injection under fluoroscopic control. Formulation 4 containing 40% w/v strontium carbonate showed the best radio-opacity (qualitative assessment by visual inspection).

Leakage: quantification of leakage volumes was visually estimated from the fluoroscopy images. Venous leakage was observed in 34 out of 100 injections (34%), where 16 consisted of less than 0.2 mL, 11 between 0.2 and 0.4 mL and 7 between 0.5 and 1.5 mL. Only one leakage episode (1%) of about 0.5 mL was symptomatic and caused cardiac distress (bradycardia and arrest). The sheep however, recovered immediately post-operatively and completed the followup without further complications. Leakage into the medullary cavity occurred in 19 cases (19%), leakage out of the cortical bone occurred in 4 cases (4%) and in one case, the type of leakage was not reported (1%). Leakage episodes into the medullary cavity and out of the cortical bone were all asymptomatic and were associated with incorrect positioning of the needle within the trabecular bone rather than with the performance of the Vertebral Augmentation Material. The formulation that showed the least number of leaks contained the highest amount of powder filler (40% w/v strontium carbonate), whereas the one comprising no filler showed the highest amount of leaks. In general, injections in the tibia and femur showed similar results in terms of number of leaks. Occurrence of leakage could not be examined for the unmodified fibrin formulation, due its unsuitable radio-opacity for appropriate intra-operative fluoroscopy monitoring.

Radiography: plane radiographs were taken at 6 and 12 weeks (sacrifice) post-op. Results showed that the formulation containing TCP filler and iodinated contrast agent as well as the one containing no filler and iodinated contrast agent were not visible already at the 6-week time-point. This indicated that the iodine component diffused out of the fibrin matrix within the first 6 weeks. For the TCP-containing formulation, it is unclear whether the filler underwent significant degradation or if the similar X-ray absorbance of TCP and bone did not allow for the identification of the filler material. In contrast to these two formulations, those containing the high X-ray absorbing strontium carbonate filler and the unmodified fibrin with gold particles were still visible at 6 weeks and at sacrifice, indicating the presence of the radio-opaque agent and no significant migration of material from the injected site.

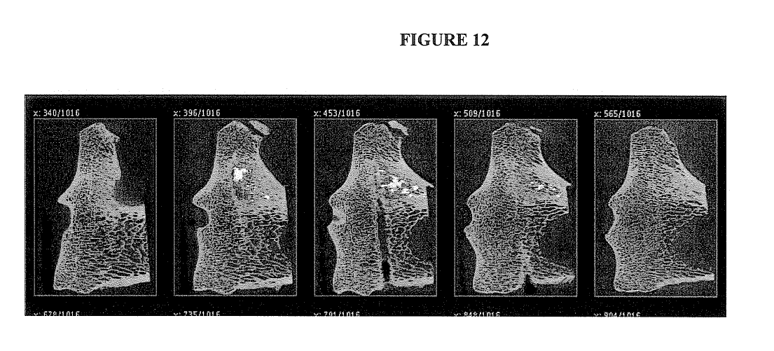

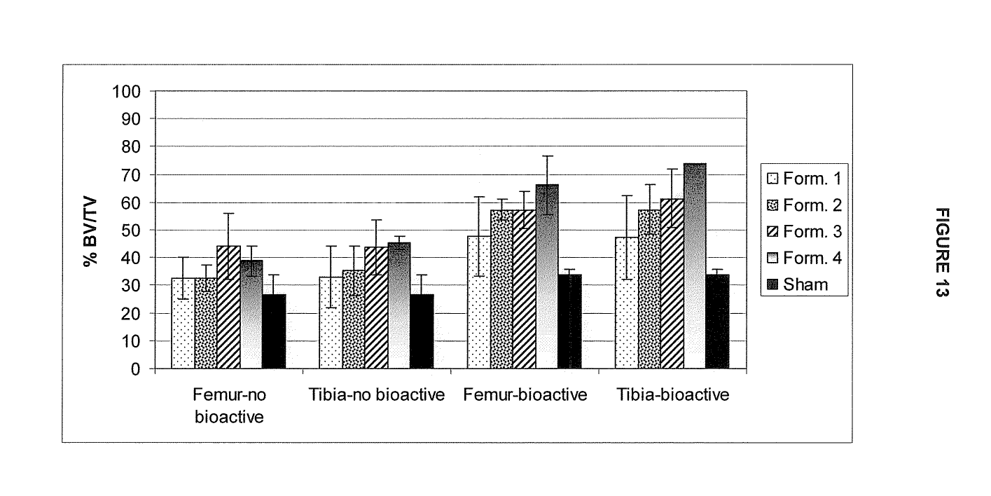

μCT:μCT images confirmed the suitability of the surgical method with respect to consistent placement of the needle and injection of the Vertebral Augmentation Material in the desired trabecular bony area. Visual inspection of the μCT two dimensional pictures indicated poor bone formation for the formulations containing TCP and no filler. Sheep treated with formulations containing TCP showed a resorption area around the injected material. In contrast, higher bone density was identified in the two formulations containing strontium carbonate. Formation of new bone and degradation of Vertebral Augmentation Material occurred inwards from the interface between material and bone towards the center of the material. Images showed the presence of residual strontium carbonate (FIG. 12). Moreover, sham treated sheep showed no significant new bone formation.

Calculation of bone volume/total volume (BV/TV) in spherical volumes of interest centered on the original position of the injection needle tip indicated a positive effect of all the Vertebral Augmentation Material formulations with respect to bone formation—with and without bioactive (TGplPTH₁₋₃₄)—when compared to sham controls. In particular, the two formulations containing strontium carbonate showed the best outcome in terms of bone formation (FIG. 13 and table 5). Furthermore, there was a tendency for higher BV/TV values in the tibias than in the femurs. It must be noted that the values for formulation 1) may be overestimated due to the incorporation of TCP in the analysis (TCP has similar X-ray absorbance to bone).

TABLE 5

| | Form. 1 | Form. 2 | Form. 3 | Form. 4 | Sham |
|---|---|---|---|---|---|
| Femur-no bioactive | n = 6 | n = 5 | n = 6 | n = 2 | n = 2 |
| Tibia-no bioactive | n = 6 | n = 4 | n = 6 | n = 2 | n = 2 |
| Femur-bioactive | n = 5 | n = 5 | n = 6 | n = 2 | |
| Tibia-bioactive | n = 6 | n = 5 | n = 6 | n = 1 | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition obtainable by mixing at least
(a) a first component comprising fibrinogen;
(b) a second component comprising thrombin, and
(c) a third component comprising strontium carbonate.

2-16. (canceled)

17. A kit of parts, comprising
a) a first container comprising fibrinogen,
b) a second container comprising thrombin, and
c) a third container comprising strontium carbonate.

18-46. (canceled)

47. The composition of claim 1, wherein the concentration of strontium carbonate is in a range from about 0.05 g/ml to about 0.60 g/ml of the total volume of the first and the second components (volume of (a)+volume of (b)).

48. The composition of claim 1, wherein the first component comprises fibrinogen in a concentration from about 25 mg/ml to about 100 mg/ml of the total volume of the first component and the second component (volume of (a)+volume of (b)), and
wherein the second component comprises thrombin in a concentration from about 1 IU to about 40 IU per ml of total volume the first component and the second component (volume of (a)+volume of (b)).

49. The composition of claim 1, wherein the composition is formed by reacting the first and the second components in the presence of a calcium source.

50. The composition of claim 1, further comprising an active agent

51. The composition of claim 50, wherein the active agent is suitable for augmenting, strengthening, supporting, repairing, rebuilding healing or filling hard tissue.

52. The composition of claim 51, wherein the active agent comprises parathyroid hormone (PTH).

53. The composition of claim 52, wherein the PTH is selected from the group consisting of PTH 1-84, PTH 1-38, PTH 1-34, PTH 1-31, PTH 1-25, and a PTH fusion peptide.

54. The composition of claim 53, wherein the PTH is a PTH fusion peptide comprising a first and a second domain, wherein the first domain comprises PTH 1-34 and the second domain comprises a substrate domain which is covalently crosslinkable to the fibrin matrix during its formation.

55. The composition of claim 52, wherein the concentration of PTH is in a range from about 0.2 mg/ml to about 5 mg/ml of the total volume of the first and the second component (volume of (a)+volume of (b)).

56. The composition of claim 1, further comprising an iodinated contrast agent.

57. The composition of claim 10, wherein the iodinated contrast agent is selected from the group consisting of diatrizoate, iodecol, iodixanol, iofratol, iogulamide, iohexyl, iomeprol, iopamidol, iotrol, ioversol, ioxaglate and metrizamide.

58. The kit of claim 17, wherein the concentration of strontium carbonate is in a range of claim 1, wherein the first component comprises fibrinogen in a concentration from about 0.05 g/ml to about 0.60 g/ml of the total volume of the first and the second component (volume of (a)+volume of (b)).

59. The kit of claim 17, wherein the first component comprises fibrinogen in a concentration from about 25 mg/ml to about 100 mg/ml of the total volume of the first component and the second component (volume of (a)+volume of (b)), and
wherein the second component comprises thrombin in a concentration from about 1 IU to about 40 IU per ml of total volume the first component and the second component (volume of (a)+volume of (b)).

60. The kit of claim 17, further comprising an active agent.

61. The kit of claim 60, wherein the active agent is suitable for augmenting, strengthening, supporting, repairing, rebuilding healing or filling hard tissue.

62. The kit of claim 61, wherein the active agent comprises parathyroid hormone (PTH).

63. The kit of claim 62, wherein the PTH is selected from the group consisting of PTH 1-84, PTH 1-38, PTH 1-34, PTH 1-31, PTH 1-25, and a PTH fusion peptide.

64. The kit of claim 63, wherein the PTH is a PTH fusion peptide comprising a first and a second domain wherein the first domain comprises PTH 1-34 and the second domain comprises a substrate domain which is covalently crosslinkable to the fibrin matrix during its formation.

65. The kit of claim 62, wherein the concentration of PTH is in a range from about 0.2 mg/ml to about 5 mg/ml of the total volume of the first and the second component (volume of (a)+volume of (b)).

66. The kit of claim 17, further comprising an iodinated contrast agent.

67. A method for treating, repairing, reducing, preventing or remodeling hard tissue in a patient, comprising providing a composition obtainable by mixing at least
(a) a first component comprising fibrinogen;
(b) a second component comprising thrombin and a
(c) third component comprising strontium carbonate.

68. The method of claim 67, wherein the hard tissue is a bone fracture.

69. The method of claim 68, wherein the treatment is a prophylactic treatment to prevent bone fractures in a vertebra.

70. A method for treating, repairing, reducing, preventing or remodeling hard tissue, comprising locally applying strontium carbonate to the hard tissue.


SEQUENCE LISTING (15 sequences)

SEQ ID NO: 1 - protein, 5 aa, Homo sapeins
GAKDV

SEQ ID NO: 2 - protein, 4 aa, Homo sapiens
KKKK

SEQ ID NO: 3 - protein, 8 aa, Homo sapiens
NQEQVSPL

SEQ ID NO: 4 - protein, 16 aa, Homo sapiens
YRGDTIGEGQQHHLGG

SEQ ID NO: 5 - protein, 6 aa, Unknown
  Sample substrate sequence for protease
LIKMKP

SEQ ID NO: 6 - protein, 6 aa, Unknown
  Sample substrate sequence for protease
NFKSQL

SEQ ID NO: 7 - protein, 8 aa, Unknown
  Sample substrate sequence for protease
GPLALTAL

SEQ ID NO: 8 - protein, 6 aa, Unknown
  Sample substrate sequence for protease
PFELRA

SEQ ID NO: 9 - protein, 5 aa, Unknown
  Sample substrate sequence for protease
ZAAFA

SEQ ID NO: 10 - protein, 8 aa, Unknown
  Sample substrate sequence for protease
GPLGIAGP

SEQ ID NO: 11 - protein, 8 aa, Unknown
  Sample substrate sequence for protease
PHYGRSGG

SEQ ID NO: 12 - protein, 9 aa, Unknown
  Sample substrate sequence for protease
PGSGRSASG

SEQ ID NO: 13 - protein, 4 aa, Homo sapiens
YKNR

SEQ ID NO: 14 - protein, 46 aa, Homo sapiens
NQEQVSPLYKNRSVSEIQLMHNLGKHLNSMERVEWLRKKLQDVHNF

SEQ ID NO: 15 - protein, 42 aa, Homo sapiens
NQEQVSPLSVSEIQLMHNLGKHLNSMERVEWLRKKLQDVHNF